SEQUENCE LISTING (74 sequences)

SEQ ID NO: 1 - DNA, 17 bp, Artificial Sequence
  Description of Artificial Sequence Synthetic oligonucleotide
ccctgccaggtaaggat

SEQ ID NO: 2 - DNA, 17 bp, Artificial Sequence
  Description of Artificial Sequence Synthetic oligonucleotide
  Description of Combined DNA/RNA Molecule Synthetic oligonucleotide
ccctgccaggtaaggat

SEQ ID NO: 3 - DNA, 17 bp, Artificial Sequence
  Description of Artificial Sequence Synthetic oligonucleotide
  Description of Combined DNA/RNA Molecule Synthetic oligonucleotide
ccctgccaggtaaggat

SEQ ID NO: 4 - DNA, 17 bp, Artificial Sequence
  Description of Artificial Sequence Synthetic oligonucleotide
  Description of Combined DNA/RNA Molecule Synthetic oligonucleotide
ccctgccaggtaaggat

SEQ ID NO: 5 - DNA, 16 bp, Artificial Sequence
  Description of Artificial Sequence Synthetic oligonucleotide
cctgccaggtaaggat

SEQ ID NO: 6 - DNA, 16 bp, Artificial Sequence
  Description of Artificial Sequence Synthetic oligonucleotide
  Description of Combined DNA/RNA Molecule Synthetic oligonucleotide
cctgccaggtaaggat

SEQ ID NO: 7 - DNA, 16 bp, Artificial Sequence
  Description of Artificial Sequence Synthetic oligonucleotide
  Description of Combined DNA/RNA Molecule Synthetic oligonucleotide
cctgccaggtaaggat

SEQ ID NO: 8 - DNA, 16 bp, Artificial Sequence
  Description of Artificial Sequence Synthetic oligonucleotide
  Description of Combined DNA/RNA Molecule Synthetic oligonucleotide
cctgccaggtaaggat

SEQ ID NO: 9 - DNA, 16 bp, Artificial Sequence
  Description of Artificial Sequence Synthetic oligonucleotide
  Description of Combined DNA/RNA Molecule Synthetic oligonucleotide
cctgccaggtaaggat

SEQ ID NO: 10 - DNA, 17 bp, Artificial Sequence
  Description of Artificial Sequence Synthetic oligonucleotide
cccctgccaggtaagga

SEQ ID NO: 11 - DNA, 17 bp, Artificial Sequence
  Description of Artificial Sequence Synthetic oligonucleotide
  Description of Combined DNA/RNA Molecule Synthetic oligonucleotide
cccctgccaggtaagga

SEQ ID NO: 12 - DNA, 17 bp, Artificial Sequence
  Description of Artificial Sequence Synthetic oligonucleotide
  Description of Combined DNA/RNA Molecule Synthetic oligonucleotide
cccctgccaggtaagga

SEQ ID NO: 13 - DNA, 17 bp, Artificial Sequence
  Description of Artificial Sequence Synthetic oligonucleotide
  Description of Combined DNA/RNA Molecule Synthetic oligonucleotide
cccctgccaggtaagga

SEQ ID NO: 14 - DNA, 16 bp, Artificial Sequence
  Description of Artificial Sequence Synthetic oligonucleotide
ccctgccaggtaagga

SEQ ID NO: 15 - DNA, 16 bp, Artificial Sequence
  Description of Artificial Sequence Synthetic oligonucleotide
  Description of Combined DNA/RNA Molecule Synthetic oligonucleotide
ccctgccaggtaagga

SEQ ID NO: 16 - DNA, 16 bp, Artificial Sequence
  Description of Artificial Sequence Synthetic oligonucleotide
  Description of Combined DNA/RNA Molecule Synthetic oligonucleotide
ccctgccaggtaagga

SEQ ID NO: 17 - DNA, 16 bp, Artificial Sequence
  Description of Artificial Sequence Synthetic oligonucleotide
  Description of Combined DNA/RNA Molecule Synthetic oligonucleotide
ccctgccaggtaagga

SEQ ID NO: 18 - DNA, 16 bp, Artificial Sequence
  Description of Artificial Sequence Synthetic oligonucleotide
  Description of Combined DNA/RNA Molecule Synthetic oligonucleotide
ccctgccaggtaagga

SEQ ID NO: 19 - RNA, 17 nt, Artificial Sequence
  Description of Artificial Sequence Synthetic oligonucleotide
ccccugccagguaagga

SEQ ID NO: 20 - RNA, 17 nt, Artificial Sequence
  Description of Artificial Sequence Synthetic oligonucleotide
ccccugccagguaagga

SEQ ID NO: 21 - RNA, 17 nt, Artificial Sequence
  Description of Artificial Sequence Synthetic oligonucleotide
ccccugccagguaagga

SEQ ID NO: 22 - RNA, 17 nt, Artificial Sequence
  Description of Artificial Sequence Synthetic oligonucleotide
ccccugccagguaagga

SEQ ID NO: 23 - DNA, 18 bp, Artificial Sequence
  Description of Artificial Sequence Synthetic oligonucleotide
  Description of Combined DNA/RNA Molecule Synthetic oligonucleotide
tccccugccagguaagga

SEQ ID NO: 24 - DNA, 18 bp, Artificial Sequence
  Description of Artificial Sequence Synthetic oligonucleotide
  Description of Combined DNA/RNA Molecule Synthetic oligonucleotide
tccccugccagguaagga

SEQ ID NO: 25 - DNA, 18 bp, Artificial Sequence
  Description of Artificial Sequence Synthetic oligonucleotide
  Description of Combined DNA/RNA Molecule Synthetic oligonucleotide
tccccugccagguaagga

SEQ ID NO: 26 - DNA, 18 bp, Artificial Sequence
  Description of Artificial Sequence Synthetic oligonucleotide
  Description of Combined DNA/RNA Molecule Synthetic oligonucleotide
tccccugccagguaagga

SEQ ID NO: 27 - RNA, 16 nt, Artificial Sequence
  Description of Artificial Sequence Synthetic oligonucleotide
cccugccagguaagga

SEQ ID NO: 28 - RNA, 16 nt, Artificial Sequence
  Description of Artificial Sequence Synthetic oligonucleotide
cccugccagguaagga

SEQ ID NO: 29 - RNA, 16 nt, Artificial Sequence
  Description of Artificial Sequence Synthetic oligonucleotide
cccugccagguaagga

SEQ ID NO: 30 - RNA, 16 nt, Artificial Sequence
  Description of Artificial Sequence Synthetic oligonucleotide
cccugccagguaagga

SEQ ID NO: 31 - DNA, 15 bp, Artificial Sequence
  Description of Artificial Sequence Synthetic oligonucleotide
  Description of Combined DNA/RNA Molecule Synthetic oligonucleotide
cctgccagguaagga

SEQ ID NO: 32 - DNA, 15 bp, Artificial Sequence
  Description of Artificial Sequence Synthetic oligonucleotide
  Description of Combined DNA/RNA Molecule Synthetic oligonucleotide
cctgccagguaagga

SEQ ID NO: 33 - RNA, 15 nt, Artificial Sequence
  Description of Artificial Sequence Synthetic oligonucleotide
ccugccagguaagga

SEQ ID NO: 34 - RNA, 15 nt, Artificial Sequence
  Description of Artificial Sequence Synthetic oligonucleotide
ccugccagguaagga

SEQ ID NO: 35 - RNA, 15 nt, Artificial Sequence
  Description of Artificial Sequence Synthetic oligonucleotide
ccugccagguaagga

SEQ ID NO: 36 - DNA, 14 bp, Artificial Sequence
  Description of Artificial Sequence Synthetic oligonucleotide
  Description of Combined DNA/RNA Molecule Synthetic oligonucleotide
ctgccagguaagga

SEQ ID NO: 37 - DNA, 14 bp, Artificial Sequence
  Description of Artificial Sequence Synthetic oligonucleotide
  Description of Combined DNA/RNA Molecule Synthetic oligonucleotide
ctgccagguaagga

SEQ ID NO: 38 - DNA, 14 bp, Artificial Sequence
  Description of Artificial Sequence Synthetic oligonucleotide
  Description of Combined DNA/RNA Molecule Synthetic oligonucleotide
ctgccagguaagga

SEQ ID NO: 39 - RNA, 14 nt, Artificial Sequence
  Description of Artificial Sequence Synthetic oligonucleotide
cugccagguaagga

SEQ ID NO: 40 - RNA, 14 nt, Artificial Sequence
  Description of Artificial Sequence Synthetic oligonucleotide
cugccagguaagga

SEQ ID NO: 41 - DNA, 13 bp, Artificial Sequence
  Description of Artificial Sequence Synthetic oligonucleotide
  Description of Combined DNA/RNA Molecule Synthetic oligonucleotide
tgccagguaagga

SEQ ID NO: 42 - DNA, 13 bp, Artificial Sequence
  Description of Artificial Sequence Synthetic oligonucleotide
  Description of Combined DNA/RNA Molecule Synthetic oligonucleotide
tgccagguaagga

SEQ ID NO: 43 - DNA, 13 bp, Artificial Sequence
  Description of Artificial Sequence Synthetic oligonucleotide
  Description of Combined DNA/RNA Molecule Synthetic oligonucleotide
tgccagguaagga

SEQ ID NO: 44 - DNA, 13 bp, Artificial Sequence
  Description of Artificial Sequence Synthetic oligonucleotide
  Description of Combined DNA/RNA Molecule Synthetic oligonucleotide
tgccagguaagga

SEQ ID NO: 45 - DNA, 18 bp, Artificial Sequence
  Description of Artificial Sequence Synthetic oligonucleotide
  Description of Combined DNA/RNA Molecule Synthetic oligonucleotide
ccccugccagguaaggat

SEQ ID NO: 46 - DNA, 18 bp, Artificial Sequence
  Description of Artificial Sequence Synthetic oligonucleotide
  Description of Combined DNA/RNA Molecule Synthetic oligonucleotide
ccccugccagguaaggat

SEQ ID NO: 47 - DNA, 18 bp, Artificial Sequence
  Description of Artificial Sequence Synthetic oligonucleotide
  Description of Combined DNA/RNA Molecule Synthetic oligonucleotide
ccccugccagguaaggat

SEQ ID NO: 48 - DNA, 18 bp, Artificial Sequence
  Description of Artificial Sequence Synthetic oligonucleotide
  Description of Combined DNA/RNA Molecule Synthetic oligonucleotide
ccccugccagguaaggat

SEQ ID NO: 49 - DNA, 17 bp, Artificial Sequence
  Description of Artificial Sequence Synthetic oligonucleotide
  Description of Combined DNA/RNA Molecule Synthetic oligonucleotide
cccugccagguaaggat

SEQ ID NO: 50 - DNA, 17 bp, Artificial Sequence
  Description of Artificial Sequence Synthetic oligonucleotide
  Description of Combined DNA/RNA Molecule Synthetic oligonucleotide
cccugccagguaaggat

SEQ ID NO: 51 - DNA, 17 bp, Artificial Sequence
  Description of Artificial Sequence Synthetic oligonucleotide
  Description of Combined DNA/RNA Molecule Synthetic oligonucleotide
cccugccagguaaggat

SEQ ID NO: 52 - DNA, 17 bp, Artificial Sequence
  Description of Artificial Sequence Synthetic oligonucleotide
  Description of Combined DNA/RNA Molecule Synthetic oligonucleotide
cccugccagguaaggat

SEQ ID NO: 53 - DNA, 17 bp, Artificial Sequence
  Description of Artificial Sequence Synthetic oligonucleotide
ttctgctggtagtggtc

SEQ ID NO: 54 - DNA, 17 bp, Artificial Sequence
  Description of Artificial Sequence Synthetic oligonucleotide
  Description of Combined DNA/RNA Molecule Synthetic oligonucleotide
ttctgctggtagtggtc

SEQ ID NO: 55 - DNA, 17 bp, Artificial Sequence
  Description of Artificial Sequence Synthetic oligonucleotide
  Description of Combined DNA/RNA Molecule Synthetic oligonucleotide
ttcugcugguaguggtc

SEQ ID NO: 56 - DNA, 17 bp, Artificial Sequence
  Description of Artificial Sequence Synthetic oligonucleotide
  Description of Combined DNA/RNA Molecule Synthetic oligonucleotide
ttcugcugguaguggtc

SEQ ID NO: 57 - DNA, 16 bp, Artificial Sequence
  Description of Artificial Sequence Synthetic oligonucleotide
  Description of Combined DNA/RNA Molecule Synthetic oligonucleotide
cctgccagguaaggat

SEQ ID NO: 58 - DNA, 16 bp, Artificial Sequence
  Description of Artificial Sequence Synthetic oligonucleotide
  Description of Combined DNA/RNA Molecule Synthetic oligonucleotide
cctgccagguaaggat

SEQ ID NO: 59 - DNA, 16 bp, Artificial Sequence
  Description of Artificial Sequence Synthetic oligonucleotide
  Description of Combined DNA/RNA Molecule Synthetic oligonucleotide
ccugccagguaaggat

SEQ ID NO: 60 - DNA, 16 bp, Artificial Sequence
  Description of Artificial Sequence Synthetic oligonucleotide
  Description of Combined DNA/RNA Molecule Synthetic oligonucleotide
ccugccagguaaggat

SEQ ID NO: 61 - DNA, 16 bp, Artificial Sequence
  Description of Artificial Sequence Synthetic oligonucleotide
  Description of Combined DNA/RNA Molecule Synthetic oligonucleotide
ccugccagguaaggat

SEQ ID NO: 62 - DNA, 15 bp, Artificial Sequence
  Description of Artificial Sequence Synthetic oligonucleotide
  Description of Combined DNA/RNA Molecule Synthetic oligonucleotide
ctgccagguaaggat

SEQ ID NO: 63 - DNA, 15 bp, Artificial Sequence
  Description of Artificial Sequence Synthetic oligonucleotide
  Description of Combined DNA/RNA Molecule Synthetic oligonucleotide
ctgccagguaaggat

SEQ ID NO: 64 - DNA, 15 bp, Artificial Sequence
  Description of Artificial Sequence Synthetic oligonucleotide
  Description of Combined DNA/RNA Molecule Synthetic oligonucleotide
ctgccagguaaggat

SEQ ID NO: 65 - DNA, 15 bp, Artificial Sequence
  Description of Artificial Sequence Synthetic oligonucleotide
  Description of Combined DNA/RNA Molecule Synthetic oligonucleotide
cugccagguaaggat

SEQ ID NO: 66 - DNA, 15 bp, Artificial Sequence
  Description of Artificial Sequence Synthetic oligonucleotide
  Description of Combined DNA/RNA Molecule Synthetic oligonucleotide
cugccagguaaggat

SEQ ID NO: 67 - DNA, 14 bp, Artificial Sequence
  Description of Artificial Sequence Synthetic oligonucleotide
  Description of Combined DNA/RNA Molecule Synthetic oligonucleotide
tgccagguaaggat

SEQ ID NO: 68 - DNA, 14 bp, Artificial Sequence
  Description of Artificial Sequence Synthetic oligonucleotide
  Description of Combined DNA/RNA Molecule Synthetic oligonucleotide
tgccagguaaggat

SEQ ID NO: 69 - DNA, 14 bp, Artificial Sequence
  Description of Artificial Sequence Synthetic oligonucleotide
  Description of Combined DNA/RNA Molecule Synthetic oligonucleotide
tgccagguaaggat

SEQ ID NO: 70 - DNA, 14 bp, Artificial Sequence
  Description of Artificial Sequence Synthetic oligonucleotide
  Description of Combined DNA/RNA Molecule Synthetic oligonucleotide
tgccagguaaggat

SEQ ID NO: 71 - DNA, 13 bp, Artificial Sequence
  Description of Artificial Sequence Synthetic oligonucleotide
  Description of Combined DNA/RNA Molecule Synthetic oligonucleotide
gccagguaaggat

SEQ ID NO: 72 - DNA, 13 bp, Artificial Sequence
  Description of Artificial Sequence Synthetic oligonucleotide
  Description of Combined DNA/RNA Molecule Synthetic oligonucleotide
gccagguaaggat

SEQ ID NO: 73 - DNA, 13 bp, Artificial Sequence
  Description of Artificial Sequence Synthetic oligonucleotide
  Description of Combined DNA/RNA Molecule Synthetic oligonucleotide
gccagguaaggat

SEQ ID NO: 74 - DNA, 13 bp, Artificial Sequence
  Description of Artificial Sequence Synthetic oligonucleotide
  Description of Combined DNA/RNA Molecule Synthetic oligonucleotide
gccagguaaggat

(19) United States
(12) Patent Application Publication
(10) Pub. No.: US 2024/0018522 A1
(43) Pub. Date: Jan. 18, 2024

(54) OLIGONUCLEOTIDES USEFUL FOR MODULATION OF SPLICING

(71) Applicant: REMIX THERAPEUTICS INC., Watertown, MA (US)

(72) Inventors: Jesse Jerome Smith, Waltham, MA (US); Duncan Brown, Berkeley, CA (US); Michael W. Seiler, Belmont, MA (US); Peter Smith, Arlington, MA (US); Anant A. Agrawal, Waltham, MA (US)

(21) Appl. No.: 18/033,748

(22) PCT Filed: Oct. 26, 2021
(86) PCT No.: PCT/US2021/056671

§ 371 Date: Apr. 25, 2023

Related U.S. Application Data
(60) Provisional application No. 63/105,671, filed on Oct. 26, 2020.

(51) Int. Cl. C12N 15/113; C12N 9/78

(52) U.S. Cl. CPC C12N 15/1135; C12N 9/78; C12Y 305/04; C12N 2320/33; C12N 2310/315; C12N 2310/321; C12N 2310/3513; C12N 2310/11

(57) ABSTRACT

Described herein are oligonucleotides (e.g., single-stranded oligonucleotides) and compositions thereof for targeting a mutation in the spliceosome, such as the U 1 small nuclear RNA (snRNA), as well as related methods of use.

### CLAIM OF PRIORITY

This application claims priority to U.S. Application No. 63/105,671, filed Oct. 26, 2020, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 25, 2021, is named R2103-7009WO_SL.txt and is 22,736 bytes in size.

### BACKGROUND

Transcription of eukaryotic genes often yields precursor RNA sequences comprising alternating coding sequences (exons) and non-coding sequences (introns). These precursor RNA sequences are converted to mature RNA in part by the spliceosome, which excises introns and joins the remaining exons together. Alternative splicing mechanisms often yield numerous protein isoforms derived from the same DNA sequence, which greatly increases protein diversity. These alternative splicing pathways may often be regulated in a tissue-specific or development stage-specific manner. Disease-associated alternative splicing patterns in pre-RNAs are often mapped to changes in splice site signals or sequence motifs and regulatory splicing factors (Faustino and Cooper (2003), Genes Dev 17(4):419-37). As such, there is a need for new therapies targeting cell splicing pathways.

### SUMMARY

The present disclosure features, inter alia, oligonucleotides and compositions thereof for targeting a mutation in the spliceosome, particularly in the non-coding region of the spliceosome.

In particular, the oligonucleotides and related compositions thereof described herein are capable of targeting (e.g., binding to) the U1 small nuclear RNA (snRNA), such as an U1 snRNA comprising a sequence mutation. Recent work has shown that certain cancers (e.g., certain solid and liquid cancers) are associated with an increased prevalence of mutations in the non-coding regions of the spliceosome, e.g., the U1 snRNA (Shuai et al (2019) Nature 574(7780):707-711; Suzuki et al (2019) Nature 574(7780):712-716). Without wishing to be bound by theory, the oligonucleotides described herein may bind to U1 snRNA mutants, e.g., associated with a cancer, and result in a decrease in cancer cell proliferation.

In one aspect, the present disclosure features an oligonucleotide (e.g., a single-stranded oligonucleotide) that targets the U1 snRNA comprising one or more of the following properties: i) the oligonucleotide is between 5 and 40 nucleotides in length; ii) the oligonucleotide comprises a plurality of internucleotide phosphorothioate linkages; iii) the oligonucleotide comprises a locked nucleic acid modification; and iv) the oligonucleotide comprises a 2′O-methyl group or a 2′O-methoxyethyl group. In some embodiments, the oligonucleotide is a single-stranded oligonucleotide (e.g., an antisense oligonucleotide). In some embodiments, the single-stranded oligonucleotide targets a mutant U1 snRNA, e.g., a U1 snRNA comprising a mutation. In some embodiments, the U1 snRNA comprises at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more sequence mutations, e.g., as compared to a reference or consensus U1 snRNA sequence. In some embodiments, the U1 snRNA comprises a mutation at any one of positions 1-4, 7-28, 31-35, 38-50, 56-79, 86-101, 105-109, 112-119, 122-149, and 155-164, e.g., as compared to a reference or consensus U1 snRNA sequence. In some embodiments, the U1 snRNA comprises a plurality of mutations at any of positions 1-4, 7-28, 31-35, 38-50, 56-79, 86-101, 105-109, 112-119, 122-149, and 155-164, e.g., as compared to a reference or consensus U1 snRNA sequence. In an embodiment, binding of the single-stranded oligonucleotide to the U1 snRNA modulates the binding of the U1 snRNA to a target RNA (e.g., a pre-mRNA, mRNA). In an embodiment, the modulating of binding of the U1 snRNA to a target RNA comprises a reduction in binding of the U1 snRNA to the target RNA (e.g., by about 5%, 10%, 20%, 25%, 30%, 40%, 50%, 60%, 70%, 75%, 80%, 90%, 95%, 99%, or more).

In another aspect, the present disclosure features a single-stranded oligonucleotide of Formula (I):

X¹-N¹-N²-N³-N⁴-N⁵-N⁶-N⁷-N⁸-N⁹-N¹⁰-N¹¹-N¹²-(N¹³)ⱼ-(N¹⁴)ₖ-(N¹⁵)ₗ-(N¹⁶)ₘ-(N¹⁷)ₙ-(N¹⁸)ₒ-(N¹⁹)ₚ-(N²⁰)_{q}-X² (I),

or a pharmaceutically acceptable salt thereof, wherein each of X¹ and X² is independently hydrogen, C₁-C₁₂ alkyl, C₁-C₁₂ heteroalkyl, halo, —OR^{A}, —O—(C₁-C₆ alkyl), —O—(C₁-C₆ heteroalkyl), —N(R^{B})(R^{C}), —C(O)N(R^{B})(R^{C}), —N(R^{B})C(O)R^{D}, a phosphate group (e.g., a monophosphate group, a diphosphate group, a triphosphate group) or a phosphorothioate group; each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ is independently absent or a nucleotide (e.g., a modified nucleotide or an unmodified nucleotide); each of R^{A}, R^{B}, R^{C}, and R^{D} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of j, k, l, m, n, o, p, and q is independently an integer between 0 and 5; and x is 0, 1, or 2; wherein one or more of N¹, N², N³, N⁴, N⁵, N⁶, N⁷, N⁸, N⁹, N¹⁰, N¹¹, and N¹² are linked together via an internucleotide phosphorothioate linkage; and one or more of N¹, N², N³, N⁴, N⁵, N⁶, N⁷, N⁸, N⁹, N¹⁰, N¹¹, and N¹² comprise a locked nucleic acid modification, a 2′O-methyl group, or a 2′O-methoxyethyl group.

In an embodiment, each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ is selected from adenosine, cytidine, thymidine, guanosine, and uridine, or a modified form thereof. In an embodiment, the bond between at least 2, 3, 4, 5, 6, 7, 8, 9, 10, 11, or all of N¹, N², N³, N⁴, N⁵, N⁶, N⁷, N⁸, N⁹, N¹⁰, N¹¹, and N¹² is a phosphorothioate bond. In an embodiment, one of N¹, N², N³ comprises a locked nucleic acid modification. In an embodiment, one of N¹¹, N¹², N¹³, N¹⁴, N⁵, N¹⁶, N¹⁷, and N¹⁸ comprises a locked nucleic acid modification. In an embodiment, one of N¹, N², N³ comprises a 2′O-methyl group. In an embodiment, one of N¹¹, N¹², N¹³, N¹⁴, N⁵, N¹⁶, N¹⁷, and N¹⁸ comprises a 2′O-methyl group. In an embodiment, one of N¹, N², N³ comprises a 2′O-methoxyethyl group. In an embodiment, one of N¹¹, N¹², N¹³, N¹⁴, N⁵, N¹⁶, N¹⁷, and N¹⁸ comprises a 2′O-methoxyethyl group.

In some embodiments, the single-stranded oligonucleotide targets a mutant U1 snRNA, e.g., a U1 snRNA comprising a mutation. In some embodiments, binding of the single-stranded oligonucleotide to the U1 snRNA mediates the degradation (e.g., RNAse degradation) of a U1 snRNA target nucleotide (e.g., a pre-RNA, e.g., a pre-mRNA). In some embodiments, binding of the single-stranded oligonucleotide to the U1 snRNA prevents binding of a U1 snRNA target nucleotide (e.g., a pre-RNA, e.g., a pre-mRNA) to the U1 snRNA. Binding of the single-stranded oligonucleotide, e.g., as described herein, to the U1 snRNA may result a reduction of binding of a U1 snRNA target nucleotide (e.g., a pre-RNA, e.g., a pre-mRNA) to the U1 snRNA by about 5%, 10%, 20%, 25%, 30%, 40%, 50%, 60%, 70%, 75%, 80%, 90%, 95%, 99% or more.

In another aspect, the present disclosure features a method of modulating, e.g., inhibiting, the U1 snRNA processing of a target RNA (e.g., an RNA which encodes an ORF, mRNA, or a pre-mRNA) comprising contacting the target RNA with a single-stranded oligonucleotide, wherein the single-stranded oligonucleotide: a) has sufficient homology with the target RNA to hybridize under physiological conditions; b) mediates cleavage of the target or hinders binding of the target RNA with another polynucleotide (e.g., RNA or DNA), e.g., a U1 snRNA component; and/or c) modulates the sequence of the target RNA. In some embodiments, the method comprises one of a), b), and c). In some embodiments, the method comprises a). In some embodiments, the method comprises b). In some embodiments, the method comprises a) and b). In some embodiments, the method comprises c). In some embodiments, the method comprises two of a), b), and c). In some embodiments, the modulating, e.g., inhibition, of the U1 snRNA comprises modulating the splicing of a nucleic acid. In some embodiments, the modulating, e.g., inhibition, of the U1 snRNA comprises modulating, e.g., preventing, the interaction between the U1 snRNA and a target RNA. In some embodiments, the modulating, e.g., inhibition, of the U1 snRNA comprises mediating degradation of a target RNA by a nuclease (e.g., RNAse H).

In another aspect, the present disclosure features a method of treating a disease or disorder in a subject comprising administering to the subject a single-stranded oligonucleotide described herein. Exemplary diseases and disorders include cancer, e.g., chronic lymphocytic leukemia, hepatocellular carcinoma, non-Hodgkin lymphoma (B cell non-Hodgkin lymphoma), bladder cancer, medulloblastoma, or pancreatic adenocarcinoma. In some embodiments, the single-stranded oligonucleotide: i) is between 5 and 40 nucleotides in length; ii) comprises a plurality of internucleotide phosphorothioate linkages; iii) comprises a locked nucleic acid modification; and/or iv) comprises a 2′O-methyl group or a 2′O-methoxyethyl group. In some embodiments, the single-stranded oligonucleotide is between 5 and 40 nucleotides in length. In some embodiments, the single-stranded oligonucleotide comprises a plurality of internucleotide phosphorothioate linkages (e.g., 2, 3, 4, 5, 6, 7, 8, 9, 10, 11, 12, 13, 14, 15, 16, 17, 18, or more internucleotide phosphorothioate linkages). In some embodiments, the single-stranded oligonucleotide comprises a locked nucleic acid modification. In some embodiments, the single-stranded oligonucleotide comprises a 2′O-methyl group or a 2′O-methoxyethyl group. In some embodiments, the single-stranded oligonucleotide targets a mutant U1 snRNA, e.g., a U1 snRNA comprising a mutation. In some embodiments, the U1 snRNA comprises at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more sequence mutations, e.g., as compared to a reference or consensus U1 snRNA sequence.

The details of one or more embodiments of the invention are set forth herein. Other features, objects, and advantages of the invention will be apparent from the Detailed Description, the Examples, and the Claims.

### BRIEF DESCRIPTION OF DRAWINGS

FIG. 1 is a graph showing the results of a splicing analysis comprising a panel of exemplary single-stranded oligonucleotides in a mutant U1 cell line (JHH7). As shown, the expression of a mutant U1 snRNA leads to aberrant splicing of exons with 5′ splice site sequences that match the mutant U1 snRNA 5′ splice site recognition sequence. Efficacy of single-stranded oligonucleotides toward a mutant U1 correlates with an increase in the expression of the canonical junction (CJ) of ATXN1L and a decrease in the expression of the alternative junction (AJ).

### DETAILED DESCRIPTION

Alternative splicing pathways have been linked to the progression of numerous diseases, such as cancer. While some alternative splicing pathways are driven by mutations in protein-coding genes, such as the splicing factor 3b subunit 1 (SF3B1), recent work has implicated mutations in the non-coding components of the spliceosome as a driver of alternative splicing pathways in certain diseases (Shuai et al). Described herein are oligonucleotides (e.g., single-stranded oligonucleotides) and related compositions thereof for targeting non-coding components of the spliceosome, particularly those components bearing sequence mutations.

### Definitions

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “acquire” or “acquiring” as the terms are used herein, refer to obtaining possession of a physical entity (e.g., a sample, e.g., blood sample or liver biopsy specimen), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third-party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis, e.g., PCR (e.g., RT-PCR).

As used herein, an amount of an oligonucleotide compound (e.g., a single-stranded oligonucleotide) effective to treat a disease or disorder (e.g., a disease or disorder described herein), “therapeutically effective amount,” “effective amount” or “effective course” refers to an amount of the of an oligonucleotide compound (e.g., a single-stranded oligonucleotide) which is effective, upon single or multiple dose administration(s) to a subject, in treating a subject, or in curing, alleviating, relieving or improving a subject with a disease or disorder (e.g., cancer) beyond that expected in the absence of such treatment.

As used herein, the terms “prevent” or “preventing” as used in the context of a disorder or disease, refer to administration of an agent to a subject, e.g., the administration of an oligonucleotide compound (e.g., a single-stranded oligonucleotide) of the present disclosure to a subject, such that the onset of at least one symptom of the disorder or disease is delayed as compared to what would be seen in the absence of administration of said treatment.

As used herein, the term “subject” is intended to include human and non-human animals. Exemplary human subjects include a human patient having a disease or disorder, e.g., disease or disorder described herein (e.g., cancer), or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dogs, cats, cows, pigs, etc.

As used herein, the terms “treat” or “treating” a subject having a disorder or disease refer to subjecting the subject to a regimen, e.g., the administration of an oligonucleotide compound (e.g., a single-stranded oligonucleotide) or pharmaceutically acceptable salt thereof, or a composition comprising an oligonucleotide compound (e.g., a single-stranded oligonucleotide) or pharmaceutically acceptable salt thereof, such that at least one symptom of the disorder or disease is cured, healed, alleviated, relieved, altered, remedied, ameliorated, or improved. Treating includes administering an amount effective to alleviate, relieve, alter, remedy, ameliorate, improve or affect the disorder or disease, or the symptoms of the disorder or disease. The treatment may inhibit deterioration or worsening of a symptom of a disorder or disease. In some embodiments, treatment comprises prevention. In some embodiments, treatment does not comprise prevention.

Numerous ranges, e.g., ranges for the amount of an oligonucleotide compound (e.g., a single-stranded oligonucleotide) or a composition thereof administered per day, are provided herein. In some embodiments, the range includes both endpoints. In other embodiments, the range excludes one or both endpoints. By way of example, the range can exclude the lower endpoint. Thus, in such an embodiment, a range of 100 to 1000 mg/day, excluding the lower endpoint, would cover an amount greater than 100 that is less than or equal to 1000 mg/day.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 36 carbon atoms (“C₁-C₃₆ alkyl”). In some embodiments, an alkyl group has 1 to 32 carbon atoms (“C₁-C₃₂ alkyl”). In some embodiments, an alkyl group has 1 to 24 carbon atoms (“C₁-C₂₄ alkyl”). In some embodiments, an alkyl group has 1 to 18 carbon atoms (“C₁-C₁₈ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₂₄ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tert-amyl (C₅), n-hexyl (C₆), octyl (C₈), nonyl (C₉), decyl (C₁₀), undecyl (C₁₁), dodecyl (or lauryl) (C₁₂), tridecyl (C₁₃), tetradecyl (or myristyl) (C₁₄), pentadecyl (C₁₅), hexadecyl (or cetyl) (C₁₆), heptadecyl (C₁₇), octadecyl (or stearyl) (C₁₅), nonadecyl (C₁₉), eicosyl (or arachidyl) (C₂₀), henicosanyl (C₂₁), docosanyl (C₂₂), tricosanyl (C₂₃), and tetracosanyl (C₂₄). Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 36 carbon atoms, one or more carbon-carbon double bonds, and no triple bonds (“C₂-C₃₆ alkenyl”). In some embodiments, an alkenyl group has 2 to 32 carbon atoms (“C₂-C₃₂ alkenyl”). In some embodiments, an alkenyl group has 2 to 24 carbon atoms (“C₂-C₂₄ alkenyl”). In some embodiments, an alkenyl group has 2 to 18 carbon atoms (“C₂-C₁₈ alkenyl”). In some embodiments, an alkenyl group has 2 to 12 carbon atoms (“C₂-C₁₂ alkenyl”). In some embodiments, an alkenyl group has 2 to 8 carbon atoms (“C₂-C₈ alkenyl”). In some embodiments, an alkenyl group has 2 to 7 carbon atoms (“C₂-C₇ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). The one or more carbon double bonds can have cis or trans (or E or Z) geometry. Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₂₄ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₆), nonenyl (C₉), nonadienyl (C₉), decenyl (C₁₀), decadienyl (C₁₀), undecenyl (C₁₁), undecadienyl (C₁₁), dodecenyl (C₁₂), dodecadienyl (C₁₂), tridecenyl (C₁₃), tridecadienyl (C₁₃), tetradecenyl (C₁₄), tetradecadienyl (e.g., myristoleyl) (C₁₄), pentadecenyl (C₁₅), pentadecadienyl (C₁₅), hexadecenyl (e.g., palmitoleyl) (C₁₆), hexadecadienyl (C₁₆), heptadecenyl (C₁₇), heptadecadienyl (C₁₇), octadecenyl (e.g., oleyl) (C₁₈), octadecadienyl (e.g., linoleyl) (C₁₈), nonadecenyl (C₁₉), nonadecadienyl (C₁₉), eicosenyl (C₂₀), eicosadienyl (C₂₀), eicosatrienyl (C₂₀), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl.

As used herein, the term “alkynyl” refers to a radical of a straight-chain or branched hydrocarbon group having from 2 to 36 carbon atoms, one or more carbon-carbon triple bonds (“C₂-C₃₆ alkynyl”). In some embodiments, an alkynyl group has 2 to 32 carbon atoms (“C₂-C₃₂ alkynyl”). In some embodiments, an alkynyl group has 2 to 24 carbon atoms (“C₂-C₂₄ alkynyl”). In some embodiments, an alkynyl group has 2 to 18 carbon atoms (“C₂-C₁₈ alkynyl”). In some embodiments, an alkynyl group has 2 to 12 carbon atoms (“C₂-C₁₂ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the terms “heteroalkyl,” “heteroalkenyl,” and “heteroalkynyl,” refer to a non-cyclic stable straight or branched alkyl, alkenyl, or alkynyl chains, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl, heteroalkenyl, or heteroalkynyl group. Exemplary heteroalkyl, heteroalkenyl, and heteroalkynyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH₂—P(O)₂—CH₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. The term “alkenylene,” by itself or as part of another substituent, means, unless otherwise stated, a divalent radical derived from an alkene.

An alkylene, alkenylene, alkynylene, or heteroalkylene group may be described as, e.g., a C₁-C₆-membered alkylene, C₁-C₆-membered alkenylene, C₁-C₆-membered alkynylene, or C₁-C₆-membered heteroalkylene, wherein the term “membered” refers to the non-hydrogen atoms within the moiety. In the case of heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written.

For example, the formula —C(O)₂R′— may represent both —C(O)₂R′— and —R′C(O)₂—. Each instance of an alkylene, alkenylene, alkynylene, or heteroalkylene group may be independently optionally substituted, i.e., unsubstituted (an “unsubstituted alkylene”) or substituted (a “substituted heteroalkylene) with one or more substituents.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “cycloalkyl” refers to a radical of a non-aromatic cyclic hydrocarbon group having from 3 to 7 ring carbon atoms (“C₃-C₇ cycloalkyl”) and zero heteroatoms in the non-aromatic ring system. In some embodiments, a cycloalkyl group has 3 to 6 ring carbon atoms (“C₃-C₆ cycloalkyl”). In some embodiments, a cycloalkyl group has 5 to 7 ring carbon atoms (“C₅-C₇ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₇ cycloalkyl groups include, without limitation, the aforementioned C₃-C₆ cycloalkyl groups as well as cycloheptyl (C₇), cycloheptenyl (C₇), cycloheptadienyl (C₇), and cycloheptatrienyl (C₇), bicyclo[2.1.1]hexanyl (C₆), bicyclo[3.1.1]heptanyl (C₇), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

As used herein, the term “halo” refers to a fluorine, chlorine, bromine, or iodine radical (i.e., —F, —Cl, —Br, and —I, respectively).

As used herein, the term “heteroaryl,” refers to an aromatic heterocycle that comprises 1, 2, 3 or 4 heteroatoms selected, independently of the others, from nitrogen, sulfur and oxygen. As used herein, the term “heteroaryl” refers to a group that may be substituted or unsubstituted. A heteroaryl may be fused to one or two rings, such as a cycloalkyl, an aryl, or a heteroaryl ring. The point of attachment of a heteroaryl to a molecule may be on the heteroaryl, cycloalkyl, heterocycloalkyl or aryl ring, and the heteroaryl group may be attached through carbon or a heteroatom. A heteroaryl group can either be monocyclic (“monocyclic heteroaryl”) or a fused, bridged or spiro ring system such as a bicyclic system (“bicyclic heteroaryl”). Examples of heteroaryl groups include imidazolyl, furyl, pyrrolyl, thienyl, thiazolyl, isoxazolyl, isothiazolyl, thiadiazolyl, oxadiazolyl, pyridinyl, pyrimidyl, pyrazinyl, pyridazinyl, quinolyl, isoquinolinyl, indazolyl, benzoxazolyl, benzisooxazolyl, benzofuryl, benzothiazolyl, indolizinyl, imidazopyridinyl, pyrazolyl, triazolyl, oxazolyl, tetrazolyl, benzimidazolyl, benzoisothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidyl, pyrazolo[3,4]pyrimidyl or benzo(b)thienyl, each of which can be optionally substituted.

As used herein, the term “heterocyclyl” refers to non-aromatic ring system having ring carbon atoms and 1 to 4 ring heteroatoms, wherein each heteroatom is independently selected from nitrogen, oxygen, sulfur, boron, phosphorus, and silicon. As used herein, the term “heterocyclyl” refers to a group that may be substituted or unsubstituted. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, the heterocyclyl group is unsubstituted 3-10 membered heterocyclyl. In certain embodiments, the heterocyclyl group is substituted 3-10 membered heterocyclyl.

As used herein, the term “hydroxy” refers to the radical —OH.

The term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are pyrimidines. Other nucleobases that do not function as normal parts of the genetic code, are termed non-naturally occurring. In an embodiment, a nucleobase may be chemically modified, for example, with an alkyl (e.g., methyl), halo, —O— alkyl, or other modification.

As used herein, the definition of each expression, e.g., alkyl, m, n, etc., when it occurs more than once in any structure, is intended to be independent of its definition elsewhere in the same structure.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

U1 snRNA

Described herein are oligonucleotides (e.g., single-stranded oligonucleotides) and related compositions capable of binding to the spliceosome or component thereof. The spliceosome is a large complex in the nucleus of eukaryotic cells that comprises five small nuclear RNAs (snRNAs) and approximately 80 proteins. Together, these components work together to recognize and bind to a pre-RNA substrate, excise introns from the pre-RNA substrate, and join the remaining exons together to yield a processed RNA. One of the first steps in the splicing pathway involves the recognition of the pre-RNA substrate by the U1 snRNA. The human genome has 7 genes with the same canonical 164 bp U1 sequence, and more than 130 pseudogenes with variant U1 sequences. The U1 snRNA may be encoded by the RNU1-1, RNU1-2, RNU1-3, or RNU1-4 genes, as well as several pseudogenes (e.g., RNU1-27P, RNU1-28P, and RNVU1-18). In an embodiment, a single-stranded oligonucleotide (e.g., as described herein) binds to the gene product of a U1 gene or a U1 gene variant. In an embodiment, a single-stranded oligonucleotide (e.g., as described herein) binds to the gene product of the RNU1-1, RNU1-2, RNU1-3, or RNU1-4 genes. In an embodiment, a single-stranded oligonucleotide (e.g., as described herein) binds to the gene product of the RNU1-27P, RNU1-28P, or RNVU1-18 pseudogenes.

The U1 snRNA may bind to the 5′ end splice site of the pre-RNA substrate through a canonical ACUUACCUG sequence at the 5′ region of the U1 snRNA. Binding of the U1 snRNA to the pre-RNA triggers assembly of other non-snRNA associated factors to form the early (E) complex, which commits the pre-RNA to the splicing process. Recently, recurrent mutations in the U1 snRNA were identified in certain solid and hematological cancers (see, e.g., Shuai et al; Suzuki et al). These mutations were found to result in alternative splicing events, which yielded, inter alia, an upregulation of several oncogenetic drivers (e.g., CCND2, GLI2) and downregulation of tumor suppressor genes (PTCH). Over 150 U1 snRNA mutations were discovered, including the A to C and A to G somatic mutations at position 3.

The single-stranded oligonucleotides described herein may be capable of binding to the spliceosome, e.g., the U1 snRNA. In some embodiments, the U1 snRNA is a variant or mutant U1 snRNA. For example, the U1 snRNA may comprise a sequence mutation (e.g., an addition, deletion, or substitution in the sequence), e.g., as compared to a reference or consensus U1 snRNA sequence. In an embodiment, the U1 snRNA comprises a sequence addition. A sequence addition as used herein may refer to the inclusion of an additional nucleic acid base in a sequence, e.g., in the U1 snRNA sequence (e.g., an additional A, C, G, or U base). In an embodiment, the U1 snRNA comprises a sequence deletion. A sequence deletion as used herein may refer to the absence of a nucleic acid base in a sequence, e.g., in the U1 snRNA sequence (e.g., deletion of an A, C, G, or U base). In an embodiment, the U1 snRNA comprises a sequence substitution. A sequence substitution as used herein may refer to the replacement of a first nucleic acid base with a second nucleic acid base in a sequence, e.g., the U1 snRNA sequence (e.g., replacement of A, C, G, or U with another base).

In an embodiment, the U1 snRNA sequence comprises at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more sequence mutations, e.g., as compared to a reference or consensus U1 snRNA sequence. In an embodiment, the U1 snRNA comprises between 1 and 10 sequence mutations, 1 and 15 sequence mutations, 1 and 20 sequence mutations, or 1 and 30 sequence mutations.

The U1 snRNA may have a mutation at any position in the sequence (e.g., at any one of base positions 1-164 in the sequence). In an embodiment, the U1 snRNA comprises a mutation at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86. 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, or 164. In an embodiment, the U1 snRNA comprises a mutation at any one of positions 1-4, 7-28, 31-35, 38-50, 56-79, 86-101, 105-109, 112-119, 122-149, and 155-164. In an embodiment, the U1 snRNA comprises a plurality of mutations at positions 1-4, 7-28, 31-35, 38-50, 56-79, 86-101, 105-109, 112-119, 122-149, and 155-164. In some embodiments, the single-stranded oligonucleotides described herein are capable of binding to a U1 snRNA comprising a mutation at any one of positions 1-4, 7-28, 31-35, 38-50, 56-79, 86-101, 105-109, 112-119, 122-149, and 155-164. In some embodiments, the single-stranded oligonucleotides described herein are capable of binding to a U1 snRNA comprising a plurality of mutations at positions 1-4, 7-28, 31-35, 38-50, 56-79, 86-101, 105-109, 112-119, 122-149, and 155-164.

In an embodiment, the U1 snRNA comprises a mutation at any one of positions 1-4. In an embodiment, the U1 snRNA comprises a mutation at any one of positions 7-28. In an embodiment, the U1 snRNA comprises a mutation at any one of positions 31-35. In an embodiment, the U1 snRNA comprises a mutation at any one of positions 38-50. In an embodiment, the U1 snRNA comprises a mutation at any one of positions 56-79. In an embodiment, the U1 snRNA comprises a mutation at any one of positions 86-101. In an embodiment, the U1 snRNA comprises a mutation at any one of positions 105-109. In an embodiment, the U1 snRNA comprises a mutation at any one of positions 112-119. In an embodiment, the U1 snRNA comprises a mutation at any one of positions 122-149. In an embodiment, the U1 snRNA comprises a mutation at any one of positions 155-164. In an embodiment, the U1 snRNA comprises a mutation at bp position 3 or 28. In an embodiment, the U1 snRNA comprises a mutation at position 3. In an embodiment, the U1 snRNA comprises a mutation at position 28.

The single-stranded oligonucleotides described herein may bind to a region of the U1 snRNA bearing any type of secondary structural feature. For example, the U1 snRNA may adopt a particular secondary structure, such as a stem loop, bulge, internal loop, kink-turn, tetraloop, pseudoknot, or base-triple, and a mutation may occur in one or more of these secondary structure elements. In an embodiment, the U1 snRNA sequence comprises a mutation in a stem loop (e.g., stem loop I, stem loop II, stem loop III, or stem loop IV). In an embodiment, the U1 snRNA comprises a mutation in the Sm site (e.g., positions 126-133). In an embodiment, the U1 snRNA comprises a mutation in the 5′ splice-site recognition sequence, for example, at positions 1-11, 1-10, 1-9, 1-8, 1-7, 1-6, 1-5, 1-4, 1-3, 1-2, 2-11, 2-10, 2-9, 2-8, 2-7, 2-6, 2-5, 2-4, 2-3, 3-11, 3-10, 3-9, 3-8, 3-7, 3-6, 3-5, 3-4, 4-11, 4-10, 4-9, 4-8, 4-7, 4-6, 4-5, 5-11, 5-10, 5-9, 5-8, 5-7, 5-6, 6-11, 6-10, 6-9, 6-8, 6-7, 7-11, 7-10, 7-9, 7-8, 8-11, 8-10, 8-9, 9-11, 9-10, or 10-11. In an embodiment, the U1 snRNA comprises a mutation in a region binding to another spliceosome component, such as the U2 snRNP or U6 snRNA. In an embodiment, the U1 snRNA comprises a mutation in a region responsible for regulating polyadenylation site selection. In an embodiment, other components of the spliceosome may also comprise a mutation.

In an embodiment, a U1 snRNA mutation co-occurs with another mutation, for example, a mutation of the TERT promoter or DDX3X (see table 2 in Suzuki). In an embodiment, a U1 snRNA mutation co-occurs with a mutation in the TP53 signaling pathway, the sonic hedgehog signaling pathway, chromatin modification machinery, a transcriptional regulation component, cell signaling component, RNA metabolism component, protein modification component, or protein metabolism component. In an embodiment, a U1 snRNA mutation co-occurs with a mutation in TP53, pTERT, PPMID, PTCH1, SMO, SUFU, KMT2D, CREBBP, KMT2C, GSE1, BCOR, GLI2, MYCN, TCF4, LHX1, LDB1, PAX6, ZIC1, PRKAR1A, PIK3CA, CDKN2A, DDX3X, FBCW7, or UBE2D2.

### Single-Stranded Oligonucleotides

The present disclosure features oligonucleotides, inter alia, capable of modulating RNA splicing by targeting a variant or mutant component of the spliceosome, such as a variant or mutant U1 snRNA. In some embodiments, the oligonucleotide is a single-stranded oligonucleotide. In some embodiments, the single-stranded oligonucleotide comprises one or more of the following properties: i) the single-stranded oligonucleotide is between is between 10 and 20 nucleotides in length; ii) the single-stranded oligonucleotide comprises a plurality of internucleotide phosphorothioate linkages; iii) the single-stranded oligonucleotide comprises a locked nucleic acid modification; and iv) the single-stranded oligonucleotide comprises a 2′-O-methoxyethyl modification.

The single-stranded oligonucleotide may be between 5 and 40 nucleotides in length. In some embodiments, the single-stranded oligonucleotide is between 5 and 35 nucleotides in length, e.g., between 5 and 30, 5 and 25, 5 and 20, 5 and 15, 5 and 14, 5 and 13, 5 and 12, 5 and 11, or 5 and 10 nucleotides in length. In some embodiments, the single-stranded oligonucleotide is between 10 and 30 nucleotides in length, e.g., 10 and 25, 10 and 20, 10 and 19, 10 and 18, 10 and 17, 10 and 16, 10 and 15, 10 and 14, 10 and 13, or 10 and 12 nucleotides in length. In some embodiments, the single-stranded oligonucleotide is between 10 and 20 nucleotides in length. In some embodiments, the single-stranded oligonucleotide is between 13 and 18 nucleotides in length.

The single-stranded oligonucleotide, e.g., as described herein, may be greater than 5 nucleotides in length. For example, the single-stranded oligonucleotide may be greater than 6, 7, 8, 9, 10, 11, 12, 13, 14, 15, 16, 17, or 18 nucleotides in length. In some embodiments, the single-stranded oligonucleotide is greater than 10 nucleotides in length. In some embodiments, the single-stranded oligonucleotide is greater than 12 nucleotides in length. In some embodiments, the single-stranded oligonucleotide is greater than 13 nucleotides in length.

The single-stranded oligonucleotide may comprise a non-natural internucleotide linkage, such as a phosphorothioate, phosphorodithioate, methyl phosphonate, vinylphosphonate, boron phosphonate, phosphoroamidate, or phosohonoacetate internucleotide linkage. In an embodiment, the single-stranded linkage comprises at least 1 non-natural internucleotide linkage. In an embodiment, the single-stranded oligonucleotide comprises a plurality of non-natural internucleotide linkages, e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 non-natural internucleotide linkages. In an embodiment, the single-stranded oligonucleotide comprises between 5 and 20 non-natural internucleotide linkages, e.g., between 5 and 19, 5 and 18, 5 and 17, 5 and 16, 5 and 15, 10 and 20, 10 and 19, 10 and 18, 10 and 17, 10 and 16, 10 and 15, 10 and 14, 10 and 13, 10 and 12, 13 and 20, 13 and 19, 13 and 18, 13 and 17, 13 and 16, 13 and 15 non-natural internucleotide linkages.

In some embodiments, the single-stranded oligonucleotide comprises a phosphorothioate internucleotide linkage. In an embodiment, the single-stranded linkage comprises at least 1 phosphorothioate internucleotide linkage. In an embodiment, the single-stranded oligonucleotide comprises a plurality of phosphorothioate internucleotide linkages, e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 phosphorothioate internucleotide linkages. In an embodiment, the single-stranded oligonucleotide comprises between 5 and 20 phosphorothioate internucleotide linkages, e.g., between 5 and 19, 5 and 18, 5 and 17, 5 and 16, 5 and 15, 10 and 20, 10 and 19, 10 and 18, 10 and 17, 10 and 16, 10 and 15, 10 and 14, 10 and 13, 10 and 12, 13 and 20, 13 and 19, 13 and 18, 13 and 17, 13 and 16, 13 and 15 phosphorothioate internucleotide linkages.

In some embodiments, the single-stranded oligonucleotide comprises no non-natural internucleotide linkages. In these cases, each internucleotide linkage in the single-stranded oligonucleotide is a phosphate linkage.

The single-stranded oligonucleotide may comprise a nucleotide modification, such as a modified base, a modified sugar, a linker, a spacer, a conjugate, a label (e.g., a fluorescent dye, a radiolabel), a quencher, or a targeting moiety. In some embodiments, the single-stranded oligonucleotide comprises 1 nucleotide modification. In some embodiments, the single-stranded oligonucleotide comprises a plurality of nucleotide modifications, e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotide modifications. The nucleotide modification may be present on a single nucleotide within the single-stranded oligonucleotide sequence or on a plurality of nucleotides within the sequence. In an embodiment, 1 of the nucleotides within the single-stranded oligonucleotide sequence comprises a nucleotide modification. In an embodiment, a plurality of nucleotides within the single-stranded oligonucleotide sequence comprises a nucleotide modification. The nucleotide modification may be present on 1, 2, 3, 4, 5, 6, 7, 6, 8, 9, 10, 11, 12, 13, 14, 15, 16, 17, 18, 19, 20, or more nucleotides within the single-stranded oligonucleotide sequence. The nucleotide modification may be present on 100%, 95%, 90%, 85%, 80%, 75%, 70%, 65%, 60%, 55%, 50%, 45%, 40%, 35%, 30%, 25%, 20%, 15%, 10%, 5%, 1% of the nucleotides within the single-stranded oligonucleotide sequence. In an embodiment, the nucleotide modification is present on 100% of the nucleotides within the single-stranded oligonucleotide sequence. In an embodiment, the nucleotide modification is present on 1, 2, 3, 4, or 5 nucleotides at the 5′-terminal portion of the single-stranded oligonucleotide. In an embodiment, the nucleotide modification is present on 1, 2, 3, 4, or 5 nucleotides at the 3′-terminal portion of the single-stranded oligonucleotide. In an embodiment, the nucleotide modification is present on 1, 2, 3, 4, or 5 nucleotides at both of the 3′ and 5′-terminal portions of the single-stranded oligonucleotide.

In an embodiment, the single-stranded oligonucleotide comprises a modified nucleobase. A modified nucleobase may comprise a natural or non-natural (e.g., synthetic) chemical modification to a naturally occurring nucleobase. For example, a modified nucleobase may comprise a modified adenine, a modified guanine, a modified cytosine, a modified uracil, or a modified thymine. Exemplary modified bases include 2-aminopurine, 2,6-diaminopurine, 5′-bromo-deoxyuridinyl, 8-aza-7-deazaguanosine, 5-hydroxybutynl-2′deoxyuridine, 5-nitroindole, 5-methyl-deoxycytidine, and hydroxymethyl-deoxycytidine. In an embodiment, the modified nucleobase is an inverted nucleobase, in which the nucleobase is bound to the remainder of the single-stranded oligonucleotide in a reversed manner, e.g., an inverted thymine, cytosine, adenine, guanine, or uracil.

In some embodiments, the single-stranded oligonucleotide comprises 1 modified nucleobase. In some embodiments, the single-stranded oligonucleotide comprises a plurality of modified nucleobases, e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified nucleobases. The modified nucleobase may be present in a single nucleotide within the single-stranded oligonucleotide sequence or in a plurality of nucleotides within the sequence. The modified nucleobase may be present in 100%, 95%, 90%, 85%, 80%, 75%, 70%, 65%, 60%, 55%, 50%, 45%, 40%, 35%, 30%, 25%, 20%, 15%, 10%, 5%, 1% of the nucleotides within the single-stranded oligonucleotide sequence. In an embodiment, the modified nucleobase is present in 100% of the nucleotides within the single-stranded oligonucleotide sequence. In an embodiment, the modified nucleobase is present in 1, 2, 3, 4, or 5 nucleotides at the 5′-terminal portion of the single-stranded oligonucleotide. In an embodiment, the modified nucleobase is present in 1, 2, 3, 4, or 5 nucleotides at the 3′-terminal portion of the single-stranded oligonucleotide. In an embodiment, the modified nucleobase is present in 1, 2, 3, 4, or 5 nucleotides at both of the 3′ and 5′-terminal portions of the single-stranded oligonucleotide.

In some embodiments, the single-stranded oligonucleotide comprises a modified sugar, such as a modified deoxyribose or modified ribose. A modified sugar may comprise a natural or non-natural (e.g., synthetic) chemical modification to the sugar moiety (e.g., ribose) within a nucleotide. The modification may be present at any available position on the sugar (e.g., ribose ring), such as the 1′, 2′, 3′, 4′, or 5′ position, and each sugar may comprise a single modification or a plurality of modifications (e.g., 2, 3, 4, 5, 6, 7, or 8 modifications). Exemplary sugar modifications include 2′O-methyl groups, 2′O-methoxyethyl groups, a halogen (e.g., fluoro, bromo, iodo, or chloro), amino groups, azido groups, deoxy groups, cyclic groups, or bridged groups.

In some embodiments, the single-stranded oligonucleotide comprises 1 modified sugar. In some embodiments, the single-stranded oligonucleotide comprises a plurality of modified sugars, e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified sugars. The modified sugar may be present in a single nucleotide within the single-stranded oligonucleotide sequence or in a plurality of nucleotides within the sequence. The modified sugar may be present in 100%, 95%, 90%, 85%, 80%, 75%, 70%, 65%, 60%, 55%, 50%, 45%, 40%, 35%, 30%, 25%, 20%, 15%, 10%, 5%, 1% of the nucleotides within the single-stranded oligonucleotide sequence. In an embodiment, the modified sugar is present in 100% of the nucleotides within the single-stranded oligonucleotide sequence. In an embodiment, the modified sugar is present in 1, 2, 3, 4, or 5 nucleotides at the 5′-terminal portion of the single-stranded oligonucleotide. In an embodiment, the modified sugar is present in 1, 2, 3, 4, or 5 nucleotides at the 3′-terminal portion of the single-stranded oligonucleotide. In an embodiment, the modified sugar is present in 1, 2, 3, 4, or 5 nucleotides at both of the 3′ and 5′-terminal portions of the single-stranded oligonucleotide.

The modified sugar may be a 2′O-methyl group or a 2′O-methoxyethyl group. In an embodiment, the single-stranded oligonucleotide comprises a 2′O-methyl group. In an embodiment, the single-stranded oligonucleotide comprises a single 2′O-methyl group or a plurality of 2′O-methyl groups, e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 2′O-methyl groups. The 2′O-methyl group may be present in a single nucleotide within the single-stranded oligonucleotide sequence or in a plurality of nucleotides within the sequence. The 2′O-methyl group may be present in 100%, 95%, 90%, 85%, 80%, 75%, 70%, 65%, 60%, 55%, 50%, 45%, 40%, 35%, 30%, 25%, 20%, 15%, 10%, 5%, 1% of the nucleotides within the single-stranded oligonucleotide sequence. In an embodiment, the 2′O-methyl group is present in 100% of the nucleotides within the single-stranded oligonucleotide sequence. In an embodiment, the 2′O-methyl group is present in 1, 2, 3, 4, or 5 nucleotides at the 5′-terminal portion of the single-stranded oligonucleotide. In an embodiment, the 2′O-methyl group is present in 1, 2, 3, 4, or 5 nucleotides at the 3′-terminal portion of the single-stranded oligonucleotide. In an embodiment, the 2′O-methyl group is present in 1, 2, 3, 4, or 5 nucleotides at both of the 3′ and 5′-terminal portions of the single-stranded oligonucleotide.

In an embodiment, the single-stranded oligonucleotide comprises a 2′O-methoxyethyl group. In an embodiment, the single-stranded oligonucleotide comprises a single 2′O-methoxyethyl group or a plurality of 2′O-methoxyethyl groups, e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 2′O-methoxyethyl groups. The 2′O-methoxyethyl group may be present in a single nucleotide within the single-stranded oligonucleotide sequence or in a plurality of nucleotides within the sequence. The 2′O-methoxyethyl group may be present in 100%, 95%, 90%, 85%, 80%, 75%, 70%, 65%, 60%, 55%, 50%, 45%, 40%, 35%, 30%, 25%, 20%, 15%, 10%, 5%, 1% of the nucleotides within the single-stranded oligonucleotide sequence. In an embodiment, the 2′O-methoxyethyl group is present in 100% of the nucleotides within the single-stranded oligonucleotide sequence. In an embodiment, the 2′O-methoxyethyl group is present in 1, 2, 3, 4, or 5 nucleotides at the 5′-terminal portion of the single-stranded oligonucleotide. In an embodiment, the 2′O-methoxyethyl group is present in 1, 2, 3, 4, or 5 nucleotides at the 3′-terminal portion of the single-stranded oligonucleotide. In an embodiment, the 2′O-methoxyethyl group is present in 1, 2, 3, 4, or 5 nucleotides at both of the 3′ and 5′-terminal portions of the single-stranded oligonucleotide.

In an embodiment, the sugar modification is a locked nucleic acid. A “locked nucleic acid” as used herein refers to a nucleotide in which the ribose moiety is modified with a bridge connecting two positions within the ribose moiety. For example, a locked nucleic acid may have the structure in which the 2′-oxygen and the 4′-carbon of the ribose ring are bound by a methylene moiety to form a five-membered ring. This modification locks the ribose in the 3′-endo conformation and may impart increased structural stability to the oligonucleotide, such as higher hybridization melting temperature and improved resistance to nucleases. In an embodiment, the single-stranded oligonucleotide comprises a single locked nucleic acid or a plurality of locked nucleic acids, e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 locked nucleic acids. The locked nucleic acid may be present in a single nucleotide within the single-stranded oligonucleotide sequence or in a plurality of nucleotides within the sequence. The locked nucleic acid may be present in 100%, 95%, 90%, 85%, 80%, 75%, 70%, 65%, 60%, 55%, 50%, 45%, 40%, 35%, 30%, 25%, 20%, 15%, 10%, 5%, 1% of the nucleotides within the single-stranded oligonucleotide sequence. In an embodiment, the locked nucleic acid is present in 100% of the nucleotides within the single-stranded oligonucleotide sequence. In an embodiment, the locked nucleic acid is present in 1, 2, 3, 4, or 5 nucleotides at the 5′-terminal portion of the single-stranded oligonucleotide. In an embodiment, the locked nucleic acid is present in 1, 2, 3, 4, or 5 nucleotides at the 3′-terminal portion of the single-stranded oligonucleotide. In an embodiment, the locked nucleic acid is present in 1, 2, 3, 4, or 5 nucleotides at both of the 3′ and 5′-terminal portions of the single-stranded oligonucleotide.

In an embodiment, the single-stranded oligonucleotide comprises the sequence GGTA, GTAA, or TAAG (e.g., wherein each nucleotide may be modified or unmodified). In an embodiment, the single-stranded oligonucleotide comprises the sequence GGTAA or GTAAG (e.g., wherein each nucleotide may be modified or unmodified). In an embodiment, the single-stranded oligonucleotide comprises the sequence GGTAAG (e.g., wherein each nucleotide may be modified or unmodified). In an embodiment, the single-stranded oligonucleotide comprises the sequence CCCT, CCTG, CTGC, TGCC, GCCA, CCAG, CAGG, or AGGT (e.g., wherein each nucleotide may be modified or unmodified).

In another aspect, the single-stranded oligonucleotide is an oligonucleotide of Formula (I):

X¹-N¹-N²-N³-N⁴-N⁵-N⁶-N⁷-N⁸-N⁹-N¹⁰N¹¹-N¹²-(N¹³)ⱼ-(N¹⁴)ₖ-(N¹⁵)ₗ-(N¹⁶)ₘ-(N¹⁷)ₙ-(N¹⁸)ₒ-(N¹⁹)ₚ-(N²⁰)_{q}-X² (I)

or a pharmaceutically acceptable salt thereof, wherein each of X¹ and X² is independently hydrogen, C₁-C₁₂ alkyl, C₁-C₁₂ heteroalkyl, halo, —OR^{A}, —O—(C₁-C₆ alkyl), —O—(C₁-C₆ heteroalkyl), —N(R^{B})(R^{C}), —C(O)N(R^{B})(R^{C}), —N(R^{B})C(O)R^{D}, a phosphate group (e.g., a monophosphate group, a diphosphate group, a triphosphate group) or a phosphorothioate group; each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ is independently absent or nucleotide (e.g., a modified nucleotide or an unmodified nucleotide); each of R^{A}, R^{B}, R^{C}, and R^{D} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of j, k, l, m, n, o, p, and q is independently an integer between 0 and 5; and x is 0, 1, or 2; wherein one or more of N¹, N², N³, N⁴, N⁵, N⁶, N⁷, N⁸, N⁹, N¹⁰, N¹¹, and N¹² are linked together via an internucleotide phosphorothioate linkage; and one or more of N¹, N², N³, N⁴, N⁵, N⁶, N⁷, N⁸, N⁹, N¹⁰, N¹¹, and N¹² comprise a locked nucleic acid modification, a 2′O-methyl modification, or a 2′O-methoxyethyl modification.

In an embodiment, each of X¹ and X² is independently hydrogen, —OR^{A}, a phosphate group (e.g., a monophosphate group), or a phosphorothioate group. In an embodiment, X¹ is hydrogen, —OR^{A}, or a phosphate group (e.g., a monophosphate group), or a phosphorothioate group. In an embodiment, X² is hydrogen.

In an embodiment, each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ is selected from adenosine, cytidine, thymidine, guanosine, and uridine, or a modified form thereof. In an embodiment, N¹ is cytidine, guanosine, or thymidine.

In an embodiment, N¹ is an unmodified nucleotide. In an embodiment, N² is cytidine, guanosine, thymidine, or a modified form thereof. In an embodiment, N³ is cytidine, guanosine, thymidine, or a modified form thereof. In an embodiment, N⁴ is cytidine, guanosine, thymidine, adenosine, or a modified form thereof. In an embodiment, N⁵ is cytidine, guanosine, thymidine, uridine, adenosine, or a modified form thereof. In an embodiment, N⁶ is cytidine, guanosine, thymidine, uridine, adenosine, or a modified form thereof. In an embodiment, N⁷ is cytidine, guanosine, thymidine, uridine, adenosine, or a modified form thereof. In an embodiment, N⁸ is cytidine, guanosine, thymidine, uridine, adenosine, or a modified form thereof. In an embodiment, N⁹ is cytidine, guanosine, thymidine, uridine, adenosine, or a modified form thereof. In an embodiment, N¹⁰ is cytidine, guanosine, thymidine, uridine, adenosine, or a modified form thereof. In an embodiment, N¹¹ is cytidine, guanosine, thymidine, uridine, adenosine, or a modified form thereof. In an embodiment, N¹² is cytidine, guanosine, thymidine, uridine, adenosine, or a modified form thereof. In an embodiment, N¹³ is cytidine, guanosine, thymidine, uridine, adenosine, or a modified form thereof. In an embodiment, N¹⁴ is cytidine, guanosine, thymidine, uridine, adenosine, or a modified form thereof. In an embodiment, N¹⁵ is cytidine, guanosine, thymidine, uridine, adenosine, or a modified form thereof. In an embodiment, N¹⁶ is cytidine, guanosine, thymidine, uridine, adenosine, or a modified form thereof. In an embodiment, N¹⁷ is cytidine, guanosine, thymidine, uridine, adenosine, or a modified form thereof. In an embodiment, N¹⁸ is cytidine, guanosine, thymidine, uridine, adenosine, or a modified form thereof. In an embodiment, N¹⁹ is cytidine, guanosine, thymidine, uridine, adenosine, or a modified form thereof.

The oligonucleotide of Formula (I) may comprise 1, 2, 3, 4, 5, 6, 7, 8, 9, 10, 11, 12, 13, 14, 15, 16, 17, 18, or more locked nucleic acid modifications. In an embodiment, the oligonucleotide comprises 1, 2, 3, 4, 5, or 6 locked nucleic acid modifications. In an embodiment, the oligonucleotide comprises 1, 2, or 3 locked nucleic acid modifications at the 3′ terminus. In an embodiment, the oligonucleotide comprises 1, 2, or 3 locked nucleic acid modifications at the 5′ terminus. In an embodiment, the oligonucleotide comprises 1, 2, or 3 locked nucleic acid modifications at the 3′ terminus and 1, 2, or 3 locked nucleic acid modifications at the 5′ terminus.

In an embodiment, one or more of N¹, N², N³, N⁴, N⁵, N⁶, N⁷, N⁸, N⁹, N¹⁰, N, N¹², N¹³, N¹⁴, N¹⁵, N¹⁶, N¹⁷, N¹⁸, N¹⁹, and N²⁰ comprises a locked nucleic acid modification. In an embodiment, one of N¹, N², N³ comprises a locked nucleic acid modification. In an embodiment, two of N¹, N², N³ comprises a locked nucleic acid modification. In an embodiment, each of N¹, N², N³ comprises a locked nucleic acid modification. In an embodiment, one of N¹¹, N¹², N¹³, N¹⁴, N⁵, N¹⁶, N¹⁷, and N¹⁸ comprises a locked nucleic acid modification. In an embodiment, two of N¹¹, N¹², N¹³, N¹⁴, N¹, N¹⁶, N¹⁷, and N¹⁸ comprises a locked nucleic acid modification. In an embodiment, each of N¹¹, N¹², N¹³, N¹⁴, N⁵, N¹⁶, N¹⁷, and N¹⁸ comprises a locked nucleic acid modification.

The oligonucleotide of Formula (I) may comprise 1, 2, 3, 4, 5, 6, 7, 8, 9, 10, 11, 12, 13, 14, 15, 16, 17, 18, or more 2′O-methyl groups. In an embodiment, the oligonucleotide comprises 1, 2, 3, 4, 5, or 6 2′O-methyl groups. In an embodiment, the oligonucleotide comprises 1, 2, or 3 2′O-methyl groups at the 3′ terminus. In an embodiment, the oligonucleotide comprises 1, 2, or 3 2′O-methyl groups at the 5′ terminus. In an embodiment, the oligonucleotide comprises 1, 2, or 3 2′O-methyl groups at the 3′ terminus and 1, 2, or 3 2′-O-methyl groups at the 5′ terminus.

In an embodiment, one or more of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ comprises a 2′O-methyl group. In an embodiment, one of N¹, N², N³ comprises a 2′O-methyl group. In an embodiment, two of N¹, N², N³ comprises a 2′O-methyl group. In an embodiment, each of N¹, N², N³ comprises a 2′O-methyl group. In an embodiment, one of N¹¹, N¹², N¹³, N¹⁴, N⁵, N¹⁶, N¹⁷, and N¹⁸ comprises a 2′O-methyl group. In an embodiment, two of N¹¹, N¹², N¹³, N¹⁴, N⁵, N¹⁶, N¹⁷, and N¹⁸ comprises a 2′O-methyl group. In an embodiment, each of N¹¹, N¹², N¹³, N¹⁴, N¹, N¹⁶, N¹⁷, and Nis comprises a 2′O-methyl group.

The oligonucleotide of Formula (I) may comprise 1, 2, 3, 4, 5, 6, 7, 8, 9, 10, 11, 12, 13, 14, 15, 16, 17, 18, or more 2′O-methoxyethyl groups. In an embodiment, the oligonucleotide comprises 1, 2, 3, 4, 5, or 6 2′O-methoxyethyl groups. In an embodiment, the oligonucleotide comprises 1, 2, or 3 2′O-methoxyethyl groups at the 3′ terminus. In an embodiment, the oligonucleotide comprises 1, 2, or 3 2′O-methoxyethyl groups at the 5′ terminus. In an embodiment, the oligonucleotide comprises 1, 2, or 3 2′O-methoxyethyl groups at the 3′ terminus and 1, 2, or 3 2′O-methoxyethyl groups at the 5′ terminus.

In an embodiment, one or more of N¹, N², N³, N⁴, N⁵, N⁶, N⁷, N⁸, N⁹, N¹⁰, N, N¹², N¹³, N¹⁴, N¹⁵, N¹⁶, N¹⁷, N¹⁸, N¹⁹, and N²⁰ comprises a 2′O-methoxyethyl group. In an embodiment, one of N¹, N², N³ comprises a 2′O-methoxyethyl group. In an embodiment, two of N¹, N², N³ comprises a 2′O-methoxyethyl group. In an embodiment, each of N¹, N², N³ comprises a 2′O-methoxyethyl group. In an embodiment, one of N¹¹, N¹², N¹³, N¹⁴, N⁵, N¹⁶, N¹⁷, and Nis comprises a 2′O-methoxyethyl group. In an embodiment, two of N¹¹, N¹², N¹³, N¹⁴, N⁵, N¹⁶, N¹⁷, and N¹⁸ comprises a 2′O-methoxyethyl group. In an embodiment, each of N¹¹, N¹², N¹³, N¹⁴, N¹, N¹⁶, N¹⁷, and N¹⁸ comprises a 2′O-methoxyethyl group.

In an embodiment, one or more of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ comprises both a locked nucleic acid and a 2′O-methyl group. In an embodiment, one or more of N¹, N², N³, N⁴, N⁵, N⁶, N⁷, N⁸, N⁹, N¹⁰, N¹¹, N¹², N¹³, N¹⁴, N¹, N¹⁶, N¹⁷, N¹⁸, N¹⁹, and N²⁰ comprises both a locked nucleic acid and a 2′O-methoxyethyl group.

In an embodiment, j is 0 or 1. In an embodiment, k is 0 or 1. In an embodiment, 1 is 0 or 1. In an embodiment, m is 0 or 1. In an embodiment, n is 0 or 1. In an embodiment, o is 0 or 1. In an embodiment, p is 0 or 1. In an embodiment, q is 0 or 1. In an embodiment, each of p and q is 0. In an embodiment, each of j, k, l, m, n, and o is 0 or 1.

In an embodiment, the oligonucleotide of Formula (I) comprises 1, 2, 3, 4, 5, 6, 7, 8, 9, 10, 11, 12, 13, 14, 15, 16, 17, or 18 phosphorothioate internucleotide linkages. In an embodiment, the oligonucleotide comprises a phosphorothioate linkage between N¹ and N², between N² and N³, between N³ and N⁴, between N⁴ and N⁵, between N⁵ and N⁶, between N⁶ and N⁷, between N⁷ and N⁸, between N⁸ and N⁹, between N⁹ and N¹⁰, between N¹¹ and N¹², between N¹² and N¹³, between N¹³ and N¹⁴, between N¹⁴ and Nis, between N¹⁵ and N¹⁶, between N¹⁶ and N¹⁷, and/or between N¹⁷ and N¹⁸.

Exemplary single-stranded oligonucleotides described herein, such as those of Formula (I), are summarized in Table 1 below. In this table, “C”, “T”, “G”, “A”, and “U” represent the naturally occurring cytidine, thymidine, guanosine, adenosine, and uridine. The “+” designation indicates the presence of a locked nucleic acid in the nucleotide which it precedes, the “m” designation indicates the presence of a 2′O-methyl group in the nucleotide which it precedes, and the “*” designation indicates the presence of a phosphorothioate internucleotide linkage in the nucleotide which it precedes.

TABLE 1
Exemplary oligonucleotide sequences

| Compound | | |
|---|---|---|
| No. | SEQ ID NO. | Oligonucleotide Sequence |
| 1 | 1 | CCCTGCCAGGTAAGGAT |
| 2 | 2 | +C*+C*+C*T*G*C*C*A*G*G*T*A*A*G*+G*+A*+T |
| 3 | 3 | +C*+C*C*T*G*C*C*A*G*G*T*A*A*G*+G*+A*+T |
| 4 | 4 | C*C*C*T*G*C*C*A*G*G*T*A*A*G*+G*+A*+T |
| 5 | 5 | CCTGCCAGGTAAGGAT |
| 6 | 6 | +C*+C*+T*G*C*C*A*G*G*T*A*A*G*+G*+A*+T |
| 7 | 7 | +C*+C*T*G*C*C*A*G*G*T*A*A*G*+G*+A*+T |
| 8 | 8 | +C*C*T*G*C*C*A*G*G*T*A*A*G*+G*+A*+T |
| 9 | 9 | C*C*T*G*C*C*A*G*G*T*A*A*G*+G*+A*+T |
| 10 | 10 | CCCCTGCCAGGTAAGGA |
| 11 | 11 | +C*+C*+C*C*T*G*C*C*A*G*G*T*A*A*+G*+G*+A |
| 12 | 12 | +C*+C*C*C*T*G*C*C*A*G*G*T*A*A*+G*+G*+A |
| 13 | 13 | C*C*C*C*T*G*C*C*A*G*G*T*A*A*+G*+G*+A |
| 14 | 14 | CCCTGCCAGGTAAGGA |
| 15 | 15 | +C*+C*+C*T*G*C*C*A*G*G*T*A*A*+G*+G*+A |
| 16 | 16 | +C*+C*C*T*G*C*C*A*G*G*T*A*A*+G*+G*+A |
| 17 | 17 | +C*C*C*T*G*C*C*A*G*G*T*A*A*+G*+G*+A |
| 18 | 18 | C*C*C*T*G*C*C*A*G*G*T*A*A*+G*+G*+A |
| 19 | 19 | +C*+C*+C*mC*mU*mG*mC*mC*mA*mG*mG*mU*mA |
| | | *mA*+G*+G*+A |
| 20 | 20 | +C*+C*mC*mC*mU*mG*mC*mC*mA*mG*mG*mU*mA |
| | | *mA*+G*+G*+A |
| 21 | 21 | +C*mC*mC*mC*mU*mG*mC*mC*mA*mG*mG*mU*mA |
| | | *mA*+G*+G*+A |
| 22 | 22 | CCCCUGCCAGGUAAGGA |
| 23 | 23 | TCCCCUGCCAGGUAAGGA |
| 24 | 24 | +T*+C*+C*mC*mC*mU*mG*mC*mC*mA*mG*mG*mU* |
| | | mA*mA*+G*+G*+A |
| 25 | 25 | +T*+C*mC*mC*mC*mU*mG*mC*mC*mA*mG*mG*mU |
| | | *mA*mA*+G*+G*+A |
| 26 | 26 | +T*mC*mC*mC*mC*mU*mG*mC*mC*mA*mG*mG*mU |
| | | *mA*mA*+G*+G*+A |
| 27 | 27 | CCCUGCCAGGUAAGGA |
| 28 | 28 | +C*+C*+C*mU*mG*mC*mC*mA*mG*mG*mU*mA*mA |
| | | *+G*+G*+A |
| 29 | 29 | +C*+C*mC*mU*mG*mC*mC*mA*mG*mG*mU*mA*mA |
| | | *+G*+G*+A |
| 30 | 30 | +C*mC*mC*mU*mG*mC*mC*mA*mG*mG*mU*mA*m |
| | | A*+G*+G*+A |
| 31 | 31 | CCTGCCAGGUAAGGA |
| 32 | 32 | +C*+C*+T*mG*mC*mC*mA*mG*mG*mU*mA*mA*+G* |
| | | +G*+A |
| 33 | 33 | CCUGCCAGGUAAGGA |
| 34 | 34 | +C*+C*mU*mG*mC*mC*mA*mG*mG*mU*mA*mA*+G |
| | | *+G*+A |
| 35 | 35 | +C*mC*mU*mG*mC*mC*mA*mG*mG*mU*mA*mA*+G |
| | | *+G*+A |
| 36 | 36 | CTGCCAGGUAAGGA |
| 37 | 37 | +C*+T*+G*mC*mC*mA*mG*mG*mU*mA*mA*+G*+G* |
| | | +A |
| 38 | 38 | +C*+T*mG*mC*mC*mA*mG*mG*mU*mA*mA*+G*+G* |
| | | +A |
| 39 | 39 | CUGCCAGGUAAGGA |
| 40 | 40 | +C*mU*mG*mC*mC*mA*mG*mG*mU*mA*mA*+G*+G |
| | | *+A |
| 41 | 41 | TGCCAGGUAAGGA |
| 42 | 42 | +T*+G*+C*mC*mA*mG*mG*mU*mA*mA*+G*+G*+A |
| 43 | 43 | +T*+G*mC*mC*mA*mG*mG*mU*mA*mA*+G*+G*+A |
| 44 | 44 | +T*mG*mC*mC*mA*mG*mG*mU*mA*mA*+G*+G*+A |
| 45 | 45 | CCCCUGCCAGGUAAGGAT |
| 46 | 46 | +C*+C*+C*mC*mU*mG*mC*mC*mA*mG*mG*mU*mA |
| | | *mA*mG*+G*+A*+T |
| 47 | 47 | +C*+C*mC*mC*mU*mG*mC*mC*mA*mG*mG*mU*mA |
| | | *mA*mG*+G*+A*+T |
| 48 | 48 | +C*mC*mC*mC*mU*mG*mC*mC*mA*mG*mG*mU*mA |
| | | *mA*mG*+G*+A*+T |
| 49 | 49 | CCCUGCCAGGUAAGGAT |
| 50 | 50 | +C*+C*+C*mU*mG*mC*mC*mA*mG*mG*mU*mA*mA |
| | | *mG*+G*+A*+T |
| 51 | 51 | +C*+C*mC*mU*mG*mC*mC*mA*mG*mG*mU*mA*mA |
| | | *mG*+G*+A*+T |
| 52 | 52 | +C*mC*mC*mU*mG*mC*mC*mA*mG*mG*mU*mA*m |
| | | A*mG*+G*+A*+T |
| 53 | 53 | TTCTGCTGGTAGTGGTC |
| 54 | 54 | +T*+T*+C*T*G*C*T*G*G*T*A*G*T*G*+G*+T*+C |
| 55 | 55 | TTCUGCUGGUAGUGGTC |
| 56 | 56 | +T*+T*+C*mU*mG*mC*mU*mG*mG*mU*mA*mG*mU |
| | | *mG*+G*+T*+C |
| 57 | 57 | CCTGCCAGGUAAGGAT |
| 58 | 58 | +C*+C*+T*mG*mC*mC*mA*mG*mG*mU*mA*mA*mG |
| | | *+G*+A*+T |
| 59 | 59 | CCUGCCAGGUAAGGAT |
| 60 | 60 | +C*+C*mU*mG*mC*mC*mA*mG*mG*mU*mA*mA*mG |
| | | *+G*+A*+T |
| 61 | 61 | +C*mC*mU*mG*mC*mC*mA*mG*mG*mU*mA*mA*m |
| | | G*+G*+A*+T |
| 62 | 62 | CTGCCAGGUAAGGAT |
| 63 | 63 | +C*+T*+G*mC*mC*mA*mG*mG*mU*mA*mA*mG*+G* |
| | | +A*+T |
| 64 | 64 | +C*+T*mG*mC*mC*mA*mG*mG*mU*mA*mA*mG*+G |
| | | *+A*+T |
| 65 | 65 | CUGCCAGGUAAGGAT |
| 66 | 66 | +C*mU*mG*mC*mC*mA*mG*mG*mU*mA*mA*mG*+G |
| | | *+A*+T |
| 67 | 67 | TGCCAGGUAAGGAT |
| 68 | 68 | +T*+G*+C*mC*mA*mG*mG*mU*mA*mA*mG*+G*+A* |
| | | +T |
| 69 | 69 | +T*+G*mC*mC*mA*mG*mG*mU*mA*mA*mG*+G*+A* |
| | | +T |
| 70 | 70 | +T*mG*mC*mC*mA*mG*mG*mU*mA*mA*mG*+G*+A |
| | | *+T |
| 71 | 71 | GCCAGGUAAGGAT |
| 72 | 72 | +G*+C*+C*mA*mG*mG*mU*mA*mA*mG*+G*+A*+T |
| 73 | 73 | +G*+C*mC*mA*mG*mG*mU*mA*mA*mG*+G*+A*+T |
| 74 | 74 | +G*mC*mC*mA*mG*mG*mU*mA*mA*mG*+G*+A*+T |

In an embodiment, the single-stranded oligonucleotide is selected from a single-stranded oligonucleotide provided in Table 1, e.g., one of Compound Nos. 1-74. In an embodiment, the single-stranded oligonucleotide is Compound No. 1. In an embodiment, the single-stranded oligonucleotide is Compound No. 2. In an embodiment, the single-stranded oligonucleotide is Compound No. 3. In an embodiment, the single-stranded oligonucleotide is Compound No. 4. In an embodiment, the single-stranded oligonucleotide is Compound No. 5. In an embodiment, the single-stranded oligonucleotide is Compound No. 6. In an embodiment, the single-stranded oligonucleotide is Compound No. 7. In an embodiment, the single-stranded oligonucleotide is Compound No. 8. In an embodiment, the single-stranded oligonucleotide is Compound No. 9. In an embodiment, the single-stranded oligonucleotide is Compound No. 10. In an embodiment, the single-stranded oligonucleotide is Compound No. 11. In an embodiment, the single-stranded oligonucleotide is Compound No. 12. In an embodiment, the single-stranded oligonucleotide is Compound No. 13. In an embodiment, the single-stranded oligonucleotide is Compound No. 14. In an embodiment, the single-stranded oligonucleotide is Compound No. 15. In an embodiment, the single-stranded oligonucleotide is Compound No. 16. In an embodiment, the single-stranded oligonucleotide is Compound No. 17. In an embodiment, the single-stranded oligonucleotide is Compound No. 18. In an embodiment, the single-stranded oligonucleotide is Compound No. 19. In an embodiment, the single-stranded oligonucleotide is Compound No. 20. In an embodiment, the single-stranded oligonucleotide is Compound No. 21. In an embodiment, the single-stranded oligonucleotide is Compound No. 22. In an embodiment, the single-stranded oligonucleotide is Compound No. 23. In an embodiment, the single-stranded oligonucleotide is Compound No. 24. In an embodiment, the single-stranded oligonucleotide is Compound No. 25. In an embodiment, the single-stranded oligonucleotide is Compound No. 26. In an embodiment, the single-stranded oligonucleotide is Compound No. 27. In an embodiment, the single-stranded oligonucleotide is Compound No. 28. In an embodiment, the single-stranded oligonucleotide is Compound No. 29. In an embodiment, the single-stranded oligonucleotide is Compound No. 30. In an embodiment, the single-stranded oligonucleotide is Compound No. 31. In an embodiment, the single-stranded oligonucleotide is Compound No. 32. In an embodiment, the single-stranded oligonucleotide is Compound No. 33. In an embodiment, the single-stranded oligonucleotide is Compound No. 34. In an embodiment, the single-stranded oligonucleotide is Compound No. 35. In an embodiment, the single-stranded oligonucleotide is Compound No. 36. In an embodiment, the single-stranded oligonucleotide is Compound No. 37. In an embodiment, the single-stranded oligonucleotide is Compound No. 38. In an embodiment, the single-stranded oligonucleotide is Compound No. 39. In an embodiment, the single-stranded oligonucleotide is Compound No. 40. In an embodiment, the single-stranded oligonucleotide is Compound No. 41. In an embodiment, the single-stranded oligonucleotide is Compound No. 42. In an embodiment, the single-stranded oligonucleotide is Compound No. 43. In an embodiment, the single-stranded oligonucleotide is Compound No. 44. In an embodiment, the single-stranded oligonucleotide is Compound No. 45. In an embodiment, the single-stranded oligonucleotide is Compound No. 46. In an embodiment, the single-stranded oligonucleotide is Compound No. 47. In an embodiment, the single-stranded oligonucleotide is Compound No. 48. In an embodiment, the single-stranded oligonucleotide is Compound No. 49. In an embodiment, the single-stranded oligonucleotide is Compound No. 50. In an embodiment, the single-stranded oligonucleotide is Compound No. 51. In an embodiment, the single-stranded oligonucleotide is Compound No. 52. In an embodiment, the single-stranded oligonucleotide is Compound No. 53. In an embodiment, the single-stranded oligonucleotide is Compound No. 54. In an embodiment, the single-stranded oligonucleotide is Compound No. 55. In an embodiment, the single-stranded oligonucleotide is Compound No. 56. In an embodiment, the single-stranded oligonucleotide is Compound No. 57. In an embodiment, the single-stranded oligonucleotide is Compound No. 58. In an embodiment, the single-stranded oligonucleotide is Compound No. 59. In an embodiment, the single-stranded oligonucleotide is Compound No. 60. In an embodiment, the single-stranded oligonucleotide is Compound No. 61. In an embodiment, the single-stranded oligonucleotide is Compound No. 62. In an embodiment, the single-stranded oligonucleotide is Compound No. 63. In an embodiment, the single-stranded oligonucleotide is Compound No. 64. In an embodiment, the single-stranded oligonucleotide is Compound No. 65. In an embodiment, the single-stranded oligonucleotide is Compound No. 66. In an embodiment, the single-stranded oligonucleotide is Compound No. 67. In an embodiment, the single-stranded oligonucleotide is Compound No. 68. In an embodiment, the single-stranded oligonucleotide is Compound No. 69. In an embodiment, the single-stranded oligonucleotide is Compound No. 70. In an embodiment, the single-stranded oligonucleotide is Compound No. 71. In an embodiment, the single-stranded oligonucleotide is Compound No. 72. In an embodiment, the single-stranded oligonucleotide is Compound No. 73. In an embodiment, the single-stranded oligonucleotide is Compound No. 74.

In an embodiment, the single-stranded oligonucleotide is a single-stranded oligonucleotide provided in Table 1 (e.g., any one of SEQ ID NOs: 1-74) or a variant thereof. In an embodiment, the single-stranded oligonucleotide has about 50%, 55%, 60%, 65%, 70%, 75%, 80%, 85%, 90%, 95%, 99%, or greater sequence identity to a single-stranded oligonucleotide provided in Table 1 (e.g., any one of SEQ ID NOs: 1-74). In an embodiment, the single-stranded oligonucleotide differs by about 1, 2, 3, 4, 5, 6, 7, 8, 9, 10, 11, 12, 13, 14, 15, 16, 17, 18, 19, or 20 nucleotides from a single-stranded oligonucleotide provided in Table 1 (e.g., any one of SEQ ID NOs: 1-74). In an embodiment, the single-stranded oligonucleotide differs by between 1-20, 1-15, 1-10, 1-5, 2-20, 2-15, 2-10, 2-8, 2-6, 2-4, 5-10, 10-20 oligonucleotides from a single-stranded oligonucleotide provided in Table 1 (e.g., any one of SEQ ID NOs: 1-74).

In an embodiment, the single-stranded oligonucleotide is SEQ ID NO: 1. In an embodiment, the single-stranded oligonucleotide has about 50%, 55%, 60%, 65%, 70%, 75%, 80%, 85%, 90%, 95%, 99%, or greater sequence identity to SEQ ID NO: 1. In an embodiment, the single-stranded oligonucleotide is SEQ ID NO: 2. In an embodiment, the single-stranded oligonucleotide has about 50%, 55%, 60%, 65%, 70%, 75%, 80%, 85%, 90%, 95%, 99%, or greater sequence identity to SEQ ID NO: 2. In an embodiment, the single-stranded oligonucleotide is SEQ ID NO: 3. In an embodiment, the single-stranded oligonucleotide has about 50%, 55%, 60%, 65%, 70%, 75%, 80%, 85%, 90%, 95%, 99%, or greater sequence identity to SEQ ID NO: 3. In an embodiment, the single-stranded oligonucleotide is SEQ ID NO: 4. In an embodiment, the single-stranded oligonucleotide has about 50%, 55%, 60%, 65%, 70%, 75%, 80%, 85%, 90%, 95%, 99%, or greater sequence identity to SEQ ID NO: 4. In an embodiment, the single-stranded oligonucleotide is SEQ ID NO: 5. In an embodiment, the single-stranded oligonucleotide has about 50%, 55%, 60%, 65%, 70%, 75%, 80%, 85%, 90%, 95%, 99%, or greater sequence identity to SEQ ID NO: 5. In an embodiment, the single-stranded oligonucleotide is SEQ ID NO: 6. In an embodiment, the single-stranded oligonucleotide has about 50%, 55%, 60%, 65%, 70%, 75%, 80%, 85%, 90%, 95%, 99%, or greater sequence identity to SEQ ID NO: 6. In an embodiment, the single-stranded oligonucleotide is SEQ ID NO: 7. In an embodiment, the single-stranded oligonucleotide has about 50%, 55%, 60%, 65%, 70%, 75%, 80%, 85%, 90%, 95%, 99%, or greater sequence identity to SEQ ID NO: 7. In an embodiment, the single-stranded oligonucleotide is SEQ ID NO: 8. In an embodiment, the single-stranded oligonucleotide has about 50%, 55%, 60%, 65%, 70%, 75%, 80%, 85%, 90%, 95%, 99%, or greater sequence identity to SEQ ID NO: 8. In an embodiment, the single-stranded oligonucleotide is SEQ ID NO: 9. In an embodiment, the single-stranded oligonucleotide has about 50%, 55%, 60%, 65%, 70%, 75%, 80%, 85%, 90%, 95%, 99%, or greater sequence identity to SEQ ID NO: 9. In an embodiment, the single-stranded oligonucleotide is SEQ ID NO: 10. In an embodiment, the single-stranded oligonucleotide has about 50%, 55%, 60%, 65%, 70%, 75%, 80%, 85%, 90%, 95%, 99%, or greater sequence identity to SEQ ID NO: 10. In an embodiment, the single-stranded oligonucleotide is SEQ ID NO: 11. In an embodiment, the single-stranded oligonucleotide has about 50%, 55%, 60%, 65%, 70%, 75%, 80%, 85%, 90%, 95%, 99%, or greater sequence identity to SEQ ID NO: 11. In an embodiment, the single-stranded oligonucleotide is SEQ ID NO: 12. In an embodiment, the single-stranded oligonucleotide has about 50%, 55%, 60%, 65%, 70%, 75%, 80%, 85%, 90%, 95%, 99%, or greater sequence identity to SEQ ID NO: 12. In an embodiment, the single-stranded oligonucleotide is SEQ ID NO: 13. In an embodiment, the single-stranded oligonucleotide has about 50%, 55%, 60%, 65%, 70%, 75%, 80%, 85%, 90%, 95%, 99%, or greater sequence identity to SEQ ID NO: 13. In an embodiment, the single-stranded oligonucleotide is SEQ ID NO: 14. In an embodiment, the single-stranded oligonucleotide has about 50%, 55%, 60%, 65%, 70%, 75%, 80%, 85%, 90%, 95%, 99%, or greater sequence identity to SEQ ID NO: 14. In an embodiment, the single-stranded oligonucleotide is SEQ ID NO: 15. In an embodiment, the single-stranded oligonucleotide has about 50%, 55%, 60%, 65%, 70%, 75%, 80%, 85%, 90%, 95%, 99%, or greater sequence identity to SEQ ID NO: 15. In an embodiment, the single-stranded oligonucleotide is SEQ ID NO: 16. In an embodiment, the single-stranded oligonucleotide has about 50%, 55%, 60%, 65%, 70%, 75%, 80%, 85%, 90%, 95%, 99%, or greater sequence identity to SEQ ID NO: 16. In an embodiment, the single-stranded oligonucleotide is SEQ ID NO: 17. In an embodiment, the single-stranded oligonucleotide has about 50%, 55%, 60%, 65%, 70%, 75%, 80%, 85%, 90%, 95%, 99%, or greater sequence identity to SEQ ID NO: 17. In an embodiment, the single-stranded oligonucleotide is SEQ ID NO: 18. In an embodiment, the single-stranded oligonucleotide has about 50%, 55%, 60%, 65%, 70%, 75%, 80%, 85%, 90%, 95%, 99%, or greater sequence identity to SEQ ID NO: 18. In an embodiment, the single-stranded oligonucleotide is SEQ ID NO: 19. In an embodiment, the single-stranded oligonucleotide has about 50%, 55%, 60%, 65%, 70%, 75%, 80%, 85%, 90%, 95%, 99%, or greater sequence identity to SEQ ID NO: 19. In an embodiment, the single-stranded oligonucleotide is SEQ ID NO: 20. In an embodiment, the single-stranded oligonucleotide has about 50%, 55%, 60%, 65%, 70%, 75%, 80%, 85%, 90%, 95%, 99%, or greater sequence identity to SEQ ID NO: 20. In an embodiment, the single-stranded oligonucleotide is SEQ ID NO: 21. In an embodiment, the single-stranded oligonucleotide has about 50%, 55%, 60%, 65%, 70%, 75%, 80%, 85%, 90%, 95%, 99%, or greater sequence identity to SEQ ID NO: 21. In an embodiment, the single-stranded oligonucleotide is SEQ ID NO: 22. In an embodiment, the single-stranded oligonucleotide has about 50%, 55%, 60%, 65%, 70%, 75%, 80%, 85%, 90%, 95%, 99%, or greater sequence identity to SEQ ID NO: 22. In an embodiment, the single-stranded oligonucleotide is SEQ ID NO: 23. In an embodiment, the single-stranded oligonucleotide has about 50%, 55%, 60%, 65%, 70%, 75%, 80%, 85%, 90%, 95%, 99%, or greater sequence identity to SEQ ID NO: 23. In an embodiment, the single-stranded oligonucleotide is SEQ ID NO: 24. In an embodiment, the single-stranded oligonucleotide has about 50%, 55%, 60%, 65%, 70%, 75%, 80%, 85%, 90%, 95%, 99%, or greater sequence identity to SEQ ID NO: 24. In an embodiment, the single-stranded oligonucleotide is SEQ ID NO: 25. In an embodiment, the single-stranded oligonucleotide has about 50%, 55%, 60%, 65%, 70%, 75%, 80%, 85%, 90%, 95%, 99%, or greater sequence identity to SEQ ID NO: 25. In an embodiment, the single-stranded oligonucleotide is SEQ ID NO: 26. In an embodiment, the single-stranded oligonucleotide has about 50%, 55%, 60%, 65%, 70%, 75%, 80%, 85%, 90%, 95%, 99%, or greater sequence identity to SEQ ID NO: 26. In an embodiment, the single-stranded oligonucleotide is SEQ ID NO: 27. In an embodiment, the single-stranded oligonucleotide has about 50%, 55%, 60%, 65%, 70%, 75%, 80%, 85%, 90%, 95%, 99%, or greater sequence identity to SEQ ID NO: 27. In an embodiment, the single-stranded oligonucleotide is SEQ ID NO: 28. In an embodiment, the single-stranded oligonucleotide has about 50%, 55%, 60%, 65%, 70%, 75%, 80%, 85%, 90%, 95%, 99%, or greater sequence identity to SEQ ID NO: 28. In an embodiment, the single-stranded oligonucleotide is SEQ ID NO: 29. In an embodiment, the single-stranded oligonucleotide has about 50%, 55%, 60%, 65%, 70%, 75%, 80%, 85%, 90%, 95%, 99%, or greater sequence identity to SEQ ID NO: 29. In an embodiment, the single-stranded oligonucleotide is SEQ ID NO: 30. In an embodiment, the single-stranded oligonucleotide has about 50%, 55%, 60%, 65%, 70%, 75%, 80%, 85%, 90%, 95%, 99%, or greater sequence identity to SEQ ID NO: 30. In an embodiment, the single-stranded oligonucleotide is SEQ ID NO: 31. In an embodiment, the single-stranded oligonucleotide has about 50%, 55%, 60%, 65%, 70%, 75%, 80%, 85%, 90%, 95%, 99%, or greater sequence identity to SEQ ID NO: 31. In an embodiment, the single-stranded oligonucleotide is SEQ ID NO: 32. In an embodiment, the single-stranded oligonucleotide has about 50%, 55%, 60%, 65%, 70%, 75%, 80%, 85%, 90%, 95%, 99%, or greater sequence identity to SEQ ID NO: 32. In an embodiment, the single-stranded oligonucleotide is SEQ ID NO: 33. In an embodiment, the single-stranded oligonucleotide has about 50%, 55%, 60%, 65%, 70%, 75%, 80%, 85%, 90%, 95%, 99%, or greater sequence identity to SEQ ID NO: 33. In an embodiment, the single-stranded oligonucleotide is SEQ ID NO: 34. In an embodiment, the single-stranded oligonucleotide has about 50%, 55%, 60%, 65%, 70%, 75%, 80%, 85%, 90%, 95%, 99%, or greater sequence identity to SEQ ID NO: 34. In an embodiment, the single-stranded oligonucleotide is SEQ ID NO: 35. In an embodiment, the single-stranded oligonucleotide has about 50%, 55%, 60%, 65%, 70%, 75%, 80%, 85%, 90%, 95%, 99%, or greater sequence identity to SEQ ID NO: 35. In an embodiment, the single-stranded oligonucleotide is SEQ ID NO: 36. In an embodiment, the single-stranded oligonucleotide has about 50%, 55%, 60%, 65%, 70%, 75%, 80%, 85%, 90%, 95%, 99%, or greater sequence identity to SEQ ID NO: 36. In an embodiment, the single-stranded oligonucleotide is SEQ ID NO: 37. In an embodiment, the single-stranded oligonucleotide has about 50%, 55%, 60%, 65%, 70%, 75%, 80%, 85%, 90%, 95%, 99%, or greater sequence identity to SEQ ID NO: 37. In an embodiment, the single-stranded oligonucleotide is SEQ ID NO: 38. In an embodiment, the single-stranded oligonucleotide has about 50%, 55%, 60%, 65%, 70%, 75%, 80%, 85%, 90%, 95%, 99%, or greater sequence identity to SEQ ID NO: 38. In an embodiment, the single-stranded oligonucleotide is SEQ ID NO: 39. In an embodiment, the single-stranded oligonucleotide has about 50%, 55%, 60%, 65%, 70%, 75%, 80%, 85%, 90%, 95%, 99%, or greater sequence identity to SEQ ID NO: 39. In an embodiment, the single-stranded oligonucleotide is SEQ ID NO: 40. In an embodiment, the single-stranded oligonucleotide has about 50%, 55%, 60%, 65%, 70%, 75%, 80%, 85%, 90%, 95%, 99%, or greater sequence identity to SEQ ID NO: 40. In an embodiment, the single-stranded oligonucleotide is SEQ ID NO: 41. In an embodiment, the single-stranded oligonucleotide has about 50%, 55%, 60%, 65%, 70%, 75%, 80%, 85%, 90%, 95%, 99%, or greater sequence identity to SEQ ID NO: 41. In an embodiment, the single-stranded oligonucleotide is SEQ ID NO: 42. In an embodiment, the single-stranded oligonucleotide has about 50%, 55%, 60%, 65%, 70%, 75%, 80%, 85%, 90%, 95%, 99%, or greater sequence identity to SEQ ID NO: 42. In an embodiment, the single-stranded oligonucleotide is SEQ ID NO: 43. In an embodiment, the single-stranded oligonucleotide has about 50%, 55%, 60%, 65%, 70%, 75%, 80%, 85%, 90%, 95%, 99%, or greater sequence identity to SEQ ID NO: 43. In an embodiment, the single-stranded oligonucleotide is SEQ ID NO: 44. In an embodiment, the single-stranded oligonucleotide has about 50%, 55%, 60%, 65%, 70%, 75%, 80%, 85%, 90%, 95%, 99%, or greater sequence identity to SEQ ID NO: 44. In an embodiment, the single-stranded oligonucleotide is SEQ ID NO: 45. In an embodiment, the single-stranded oligonucleotide has about 50%, 55%, 60%, 65%, 70%, 75%, 80%, 85%, 90%, 95%, 99%, or greater sequence identity to SEQ ID NO: 45. In an embodiment, the single-stranded oligonucleotide is SEQ ID NO: 46. In an embodiment, the single-stranded oligonucleotide has about 50%, 55%, 60%, 65%, 70%, 75%, 80%, 85%, 90%, 95%, 99%, or greater sequence identity to SEQ ID NO: 46. In an embodiment, the single-stranded oligonucleotide is SEQ ID NO: 47. In an embodiment, the single-stranded oligonucleotide has about 50%, 55%, 60%, 65%, 70%, 75%, 80%, 85%, 90%, 95%, 99%, or greater sequence identity to SEQ ID NO: 47. In an embodiment, the single-stranded oligonucleotide is SEQ ID NO: 48. In an embodiment, the single-stranded oligonucleotide has about 50%, 55%, 60%, 65%, 70%, 75%, 80%, 85%, 90%, 95%, 99%, or greater sequence identity to SEQ ID NO: 48. In an embodiment, the single-stranded oligonucleotide is SEQ ID NO: 49. In an embodiment, the single-stranded oligonucleotide has about 50%, 55%, 60%, 65%, 70%, 75%, 80%, 85%, 90%, 95%, 99%, or greater sequence identity to SEQ ID NO: 49. In an embodiment, the single-stranded oligonucleotide is SEQ ID NO: 50. In an embodiment, the single-stranded oligonucleotide has about 50%, 55%, 60%, 65%, 70%, 75%, 80%, 85%, 90%, 95%, 99%, or greater sequence identity to SEQ ID NO: 50. In an embodiment, the single-stranded oligonucleotide is SEQ ID NO: 51. In an embodiment, the single-stranded oligonucleotide has about 50%, 55%, 60%, 65%, 70%, 75%, 80%, 85%, 90%, 95%, 99%, or greater sequence identity to SEQ ID NO: 51. In an embodiment, the single-stranded oligonucleotide is SEQ ID NO: 52. In an embodiment, the single-stranded oligonucleotide has about 50%, 55%, 60%, 65%, 70%, 75%, 80%, 85%, 90%, 95%, 99%, or greater sequence identity to SEQ ID NO: 52. In an embodiment, the single-stranded oligonucleotide is SEQ ID NO: 53. In an embodiment, the single-stranded oligonucleotide has about 50%, 55%, 60%, 65%, 70%, 75%, 80%, 85%, 90%, 95%, 99%, or greater sequence identity to SEQ ID NO: 53. In an embodiment, the single-stranded oligonucleotide is SEQ ID NO: 54. In an embodiment, the single-stranded oligonucleotide has about 50%, 55%, 60%, 65%, 70%, 75%, 80%, 85%, 90%, 95%, 99%, or greater sequence identity to SEQ ID NO: 54. In an embodiment, the single-stranded oligonucleotide is SEQ ID NO: 55. In an embodiment, the single-stranded oligonucleotide has about 50%, 55%, 60%, 65%, 70%, 75%, 80%, 85%, 90%, 95%, 99%, or greater sequence identity to SEQ ID NO: 55. In an embodiment, the single-stranded oligonucleotide is SEQ ID NO: 56. In an embodiment, the single-stranded oligonucleotide has about 50%, 55%, 60%, 65%, 70%, 75%, 80%, 85%, 90%, 95%, 99%, or greater sequence identity to SEQ ID NO: 56. In an embodiment, the single-stranded oligonucleotide is SEQ ID NO: 57. In an embodiment, the single-stranded oligonucleotide has about 50%, 55%, 60%, 65%, 70%, 75%, 80%, 85%, 90%, 95%, 99%, or greater sequence identity to SEQ ID NO: 57. In an embodiment, the single-stranded oligonucleotide is SEQ ID NO: 58. In an embodiment, the single-stranded oligonucleotide has about 50%, 55%, 60%, 65%, 70%, 75%, 80%, 85%, 90%, 95%, 99%, or greater sequence identity to SEQ ID NO: 58. In an embodiment, the single-stranded oligonucleotide is SEQ ID NO: 59. In an embodiment, the single-stranded oligonucleotide has about 50%, 55%, 60%, 65%, 70%, 75%, 80%, 85%, 90%, 95%, 99%, or greater sequence identity to SEQ ID NO: 59. In an embodiment, the single-stranded oligonucleotide is SEQ ID NO: 60. In an embodiment, the single-stranded oligonucleotide has about 50%, 55%, 60%, 65%, 70%, 75%, 80%, 85%, 90%, 95%, 99%, or greater sequence identity to SEQ ID NO: 60. In an embodiment, the single-stranded oligonucleotide is SEQ ID NO: 61. In an embodiment, the single-stranded oligonucleotide has about 50%, 55%, 60%, 65%, 70%, 75%, 80%, 85%, 90%, 95%, 99%, or greater sequence identity to SEQ ID NO: 61. In an embodiment, the single-stranded oligonucleotide is SEQ ID NO: 62. In an embodiment, the single-stranded oligonucleotide has about 50%, 55%, 60%, 65%, 70%, 75%, 80%, 85%, 90%, 95%, 99%, or greater sequence identity to SEQ ID NO: 62. In an embodiment, the single-stranded oligonucleotide is SEQ ID NO: 63. In an embodiment, the single-stranded oligonucleotide has about 50%, 55%, 60%, 65%, 70%, 75%, 80%, 85%, 90%, 95%, 99%, or greater sequence identity to SEQ ID NO: 63. In an embodiment, the single-stranded oligonucleotide is SEQ ID NO: 64. In an embodiment, the single-stranded oligonucleotide has about 50%, 55%, 60%, 65%, 70%, 75%, 80%, 85%, 90%, 95%, 99%, or greater sequence identity to SEQ ID NO: 64. In an embodiment, the single-stranded oligonucleotide is SEQ ID NO: 65. In an embodiment, the single-stranded oligonucleotide has about 50%, 55%, 60%, 65%, 70%, 75%, 80%, 85%, 90%, 95%, 99%, or greater sequence identity to SEQ ID NO: 65. In an embodiment, the single-stranded oligonucleotide is SEQ ID NO: 66. In an embodiment, the single-stranded oligonucleotide has about 50%, 55%, 60%, 65%, 70%, 75%, 80%, 85%, 90%, 95%, 99%, or greater sequence identity to SEQ ID NO: 66. In an embodiment, the single-stranded oligonucleotide is SEQ ID NO: 67. In an embodiment, the single-stranded oligonucleotide has about 50%, 55%, 60%, 65%, 70%, 75%, 80%, 85%, 90%, 95%, 99%, or greater sequence identity to SEQ ID NO: 67. In an embodiment, the single-stranded oligonucleotide is SEQ ID NO: 68. In an embodiment, the single-stranded oligonucleotide has about 50%, 55%, 60%, 65%, 70%, 75%, 80%, 85%, 90%, 95%, 99%, or greater sequence identity to SEQ ID NO: 68. In an embodiment, the single-stranded oligonucleotide is SEQ ID NO: 69. In an embodiment, the single-stranded oligonucleotide has about 50%, 55%, 60%, 65%, 70%, 75%, 80%, 85%, 90%, 95%, 99%, or greater sequence identity to SEQ ID NO: 69. In an embodiment, the single-stranded oligonucleotide is SEQ ID NO: 70. In an embodiment, the single-stranded oligonucleotide has about 50%, 55%, 60%, 65%, 70%, 75%, 80%, 85%, 90%, 95%, 99%, or greater sequence identity to SEQ ID NO: 70. In an embodiment, the single-stranded oligonucleotide is SEQ ID NO: 71. In an embodiment, the single-stranded oligonucleotide has about 50%, 55%, 60%, 65%, 70%, 75%, 80%, 85%, 90%, 95%, 99%, or greater sequence identity to SEQ ID NO: 71. In an embodiment, the single-stranded oligonucleotide is SEQ ID NO: 72. In an embodiment, the single-stranded oligonucleotide has about 50%, 55%, 60%, 65%, 70%, 75%, 80%, 85%, 90%, 95%, 99%, or greater sequence identity to SEQ ID NO: 72. In an embodiment, the single-stranded oligonucleotide is SEQ ID NO: 73. In an embodiment, the single-stranded oligonucleotide has about 50%, 55%, 60%, 65%, 70%, 75%, 80%, 85%, 90%, 95%, 99%, or greater sequence identity to SEQ ID NO: 73. In an embodiment, the single-stranded oligonucleotide is SEQ ID NO: 74. In an embodiment, the single-stranded oligonucleotide has about 50%, 55%, 60%, 65%, 70%, 75%, 80%, 85%, 90%, 95%, 99%, or greater sequence identity to SEQ ID NO: 74.

Methods of Targeting the U1 snRNA

The present disclosure features single-stranded oligonucleotides for use in modulating splicing. In some embodiments, the method comprises modulating (e.g., increasing or decreasing) the splicing of a nucleic acid (e.g., RNA, e.g., a pre-mRNA) comprising contacting the nucleic acid with a single-stranded oligonucleotide described herein. The single-stranded oligonucleotide may modulate splicing by inhibiting the spliceosome or by activating degradation of a target nucleic acid (e.g., RNA, e.g., a pre-mRNA). Modulation comprises increasing or decreasing the activity of any component of the spliceosome, e.g., the U1 snRNA. Recent work has shown that modulation of the mutant U1 allele may reverse the alternative splicing pattern in model cell lines (see, e.g., Shuai and Suzuki). In an embodiment, modulation of the mutant U1 allele results in about 1, 2, 3, 4, 5, 6, 7, 8, 9, 10, 15, 20, 25, 30, 35, 40, 45, 50, 55, 60, 65, 70, 75, 80, 85, 90, 95, 99% or greater reversal of a splicing pattern, e.g., an alternative splicing pattern, compared to a reference value.

Modulation of the spliceosome (e.g., the U1 snRNA) with the single-stranded oligonucleotide may occur in numerous ways. In one pathway, binding of the single-stranded oligonucleotide to the U1 snRNA prevents access of a target RNA to the U1 snRNA, for example, at the target RNA recognition site. In one embodiment, binding of the single-stranded oligonucleotide to the U1 snRNA reduces access of a target RNA to the U1 snRNA by at least about 1, 5, 10, 15, 20, 25, 30, 35, 40, 45, 50, 55, 60, 65, 70, 75, 80, 85, 90, 95, 99%, or more, relative to a reference standard. In one embodiment, the target recognition site is the 5′ splice site recognition sequence in the U1 snRNA. In one embodiment, the target recognition site comprises a mutation in the 5′ splice site recognition sequence in the U1 snRNA. In an embodiment, the target recognition site within the U1 snRNA comprises positions 2-12, e.g., 3-10. In an embodiment, the target recognition site comprises positions 3-8. In an embodiment, the target recognition site comprises position 3. In an embodiment, U1 snRNA comprises a mutation at positions 2-12, e.g., 3-10. In an embodiment, U1 snRNA comprises a mutation at positions 3-8. In an embodiment, U1 snRNA comprises a mutation at position 3.

In another pathway, modulation of the spliceosome (e.g., the U1 snRNA) with the single-stranded oligonucleotide comprises mediating degradation of a target RNA. In some embodiments, binding of the single-stranded oligonucleotide to the U1 snRNA mediates the nuclease degradation of a target RNA, e.g., by RNAse H. In some embodiments, RNAse H degradation of a target RNA is increased by about 1, 5, 10, 15, 20, 25, 30, 35, 40, 45, 50, 55, 60, 65, 70, 75, 80, 85, 90, 95, 99%, or more, relative to a reference standard.

In another pathway, modulation of the spliceosome (e.g., the U1 snRNA) with the single-stranded oligonucleotide comprises mediating degradation of the U1 snRNA/single-stranded oligonucleotide complex, e.g., by a nuclease (e.g., RNAse H). In some embodiments, RNAse H degradation of the U1 snRNA/single-stranded oligonucleotide complex is increased by about 1, 5, 10, 15, 20, 25, 30, 35, 40, 45, 50, 55, 60, 65, 70, 75, 80, 85, 90, 95, 99%, or more, relative to a reference standard.

In yet another way, modulation of the spliceosome (e.g., the U1 snRNA) is affected by administration of a single-stranded oligonucleotide conjugate. In some embodiments, the single-stranded oligonucleotide is bound (e.g., covalently or non-covalently bound) to a conjugated moiety. Exemplary conjugated moieties include labels (e.g., GFP or other fluorophore), a targeting agent (e.g., biotin), and a nucleic acid editing moiety. In an embodiment, the single-stranded oligonucleotide is conjugated to an enzyme, e.g., an RNA editing enzyme. In an embodiment, the single-stranded oligonucleotide is conjugated to the catalytic domain of the RNA editing enzyme ADAR. Without wishing to be bound by theory, in an embodiment, the single-stranded oligonucleotide conjugate (e.g., the ADAR conjugate) may drive conversion of one nucleobase to another (Montiel-Gonzalez (2016) Nuc Acid Res 44(21):e157). In an embodiment, the protein conjugate is tethered to the single-stranded oligonucleotide by a linker (e.g., alkyl linker, peptide linker). In an embodiment, the single-stranded conjugate induces conversion of one nucleobase to another, e.g., adenosine to inosine.

In another aspect, the present disclosure features a method of inhibiting the U1 snRNA processing of a target RNA (e.g., an RNA which encodes an ORF, mRNA, or a pre-mRNA) comprising contacting the target RNA with a single-stranded oligonucleotide, wherein the single-stranded oligonucleotide: a) has sufficient homology with the target RNA to hybridize under physiological conditions; b) mediates cleavage of the target or hinders binding of the target RNA with another polynucleotide (e.g., RNA or DNA), e.g., a U1 snRNA component; and/or c) edits a nucleotide within the sequence of the U1 snRNA component. In some embodiments, the single-stranded oligonucleotide has sufficient homology with the target RNA to hybridize under physiological conditions. In some embodiments, the single-stranded oligonucleotide mediates cleavage of the target RNA and/or hinders binding of the target RNA with another polynucleotide (e.g., RNA or DNA), e.g., a U1 snRNA component. In some embodiments, the single-stranded oligonucleotide is capable of editing a nucleotide within the sequence of the U1 snRNA component

Modulation of splicing, e.g., by a single-stranded oligonucleotide described herein, may result in a reduction of the level of a target RNA or the level of a target protein formed. In some embodiments, the level of a target RNA is reduced by about 1, 5, 10, 15, 20, 25, 30, 35, 40, 45, 50, 55, 60, 65, 70, 75, 80, 85, 90, 95, 99%, or more, relative to a reference standard. In some embodiments, the level of a target protein is reduced by about 1, 5, 10, 15, 20, 25, 30, 35, 40, 45, 50, 55, 60, 65, 70, 75, 80, 85, 90, 95, 99%, or more, relative to a reference standard.

Modulation of splicing, e.g., by a single-stranded oligonucleotide described herein, may result in an increase in the level of a target RNA or the level of a target protein formed. In some embodiments, the level of a target RNA is increased by about 1, 5, 10, 15, 20, 25, 30, 35, 40, 45, 50, 55, 60, 65, 70, 75, 80, 85, 90, 95, 99%, or more, relative to a reference standard. In some embodiments, the level of a target protein is increased by about 1, 5, 10, 15, 20, 25, 30, 35, 40, 45, 50, 55, 60, 65, 70, 75, 80, 85, 90, 95, 99%, or more, relative to a reference standard.

Modulation of splicing, e.g., by a single-stranded oligonucleotide described herein, may result in modulation (e.g., an increase or reduction) in cell viability. For example, decreased splicing activity may slow the progression of a disease state by decreasing the level of a target RNA or target protein formed, and ultimately yield increased cell viability. In an embodiment, cell viability in a sample is increased by about 1, 5, 10, 15, 20, 25, 30, 35, 40, 45, 50, 55, 60, 65, 70, 75, 80, 85, 90, 95, 99%, or more, relative to a reference standard. In some embodiments, modulation of splicing by a single-stranded oligonucleotide described herein results in a reduction of cell viability, e.g., by about 1, 5, 10, 15, 20, 25, 30, 35, 40, 45, 50, 55, 60, 65, 70, 75, 80, 85, 90, 95, 99%, or more, relative to a reference standard. In some embodiments, cryptic splicing in a sample is reduced, e.g., by about 1, 5, 10, 15, 20, 25, 30, 35, 40, 45, 50, 55, 60, 65, 70, 75, 80, 85, 90, 95, 99%, or more, relative to a reference standard. In some embodiments, cryptic splicing in a sample is increased, e.g., by about 1, 5, 10, 15, 20, 25, 30, 35, 40, 45, 50, 55, 60, 65, 70, 75, 80, 85, 90, 95, 99%, or more, relative to a reference standard.

Modulation of splicing, e.g., by a single-stranded oligonucleotide described herein, may result in modulation (e.g., an increase or reduction) of alternative splicing of an RNA transcript. For example, a reduction of alternative splicing of an RNA transcript might lead to formation of fewer pathogenic proteins and nucleic acids. In some embodiments, a reduction of alternative splicing occurs in a specific subset of genes (, for example, cell cycle genes and ultimately yield increased cell viability. In some embodiments, alternative selection of a 5′ splice site is increased, e.g., by about 1, 5, 10, 15, 20, 25, 30, 35, 40, 45, 50, 55, 60, 65, 70, 75, 80, 85, 90, 95, 99%, or more, relative to a reference standard. In some embodiments, alternative selection of a 5′ splice site is reduced, e.g., by about 1, 5, 10, 15, 20, 25, 30, 35, 40, 45, 50, 55, 60, 65, 70, 75, 80, 85, 90, 95, 99%, or more, relative to a reference standard. In some embodiments, excision of an intron at a 5′ splice site in a target RNA is increased, e.g., by about 1, 5, 10, 15, 20, 25, 30, 35, 40, 45, 50, 55, 60, 65, 70, 75, 80, 85, 90, 95, 99%, or more, relative to a reference standard. In some embodiments, excision of an intron at a 5′ splice site in a target RNA is reduced, e.g., by about 1, 5, 10, 15, 20, 25, 30, 35, 40, 45, 50, 55, 60, 65, 70, 75, 80, 85, 90, 95, 99%, or more, relative to a reference standard.

In some embodiments, alternative selection of a 3′ splice site is increased, e.g., by about 1, 5, 10, 15, 20, 25, 30, 35, 40, 45, 50, 55, 60, 65, 70, 75, 80, 85, 90, 95, 99%, or more, relative to a reference standard. In some embodiments, alternative selection of a 3′ splice site is reduced, e.g., by about 1, 5, 10, 15, 20, 25, 30, 35, 40, 45, 50, 55, 60, 65, 70, 75, 80, 85, 90, 95, 99%, or more, relative to a reference standard. In some embodiments, excision of an intron at a 3′ splice site in a target RNA is increased, e.g., by about 1, 5, 10, 15, 20, 25, 30, 35, 40, 45, 50, 55, 60, 65, 70, 75, 80, 85, 90, 95, 99%, or more, relative to a reference standard. In some embodiments, excision of an intron at 3′ splice site in a target RNA is reduced, e.g., by about 1, 5, 10, 15, 20, 25, 30, 35, 40, 45, 50, 55, 60, 65, 70, 75, 80, 85, 90, 95, 99%, or more, relative to a reference standard.

The present disclosure further features methods for forming a complex, e.g., a complex between a splicing factor and a single-stranded oligonucleotide. In some embodiments, the splicing factor is an snRNA. In some embodiments, the splicing factor is the U1 snRNA. In some embodiments, the method further comprises contacting a splicing factor (e.g., e U1 snRNA) with a single-stranded oligonucleotide described herein. The method may be carried out in any mode, for example, it may be an in vitro, in vivo, or ex vivo method. The method may be carried out in a tube, vial, plate, well, cuvette, gel, or other container. The method may be monitored to observe signs of complex formation, e.g., by any method known in the art. For example, complex formation may be measured by chromatography (size-exclusion chromatography, hydrophobic interaction chromatography), PCR (e.g., RT-PCR), NMR, mass spectrometry, fluorescence spectroscopy, dynamic light scattering, or microscopy.

### Methods of Treatment

The present disclosure provides oligonucleotides and related compositions useful for targeting the spliceosome (e.g., the U1 snRNA), as well as for treating a disease or disorder in a subject. In an embodiment, the disease or disorder is related to (e.g., caused by) a splicing event, such as an aberrant or alternative splicing event.

In an embodiment, the disease or disorder is a proliferative disease, such as a cancer or benign neoplasm. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the invention. The cancer may be associated with a solid tumor or a liquid tumor. In an embodiment, the cancer is a cancer of a particular organ system, such as the central nervous system, peripheral nervous system, digestive system, reproductive system, respiratory system, musculoskeletal system, immune system, circulatory system, endocrine system, excretory system, or lymphatic system. In an embodiment, the cancer is a cancer of the central nervous system, digestive system, or lymphatic system.

Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstram's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In an embodiment, the subject has chronic lymphocytic leukemia, hepatocellular carcinoma, non-Hodgkin lymphoma (B cell non-Hodgkin lymphoma), bladder cancer, medulloblastoma, or pancreatic adenocarcinoma. Chronic lymphocytic leukemia (CLL) is a cancer typified by an increased production of lymphocytes in the bone marrow. CLL is often preceded by a particular type of monoclonal B-cell lymphocytosis, chronic lymphocytic leukemia/small lymphocyte lymphoma (CLL/SLL MBL). Diagnostic tests for CLL typically include analysis of blood lymphocyte levels and may also include measurement of circulating amounts of one or both of the ZAP-70 or CD38 proteins, which are CLL biomarkers. In an embodiment, the subject has CLL or is identified as having CLL. In an embodiment, the subject having CLL is administered a single-stranded oligonucleotide described herein.

Hepatocellular carcinoma is the most common form of primary liver cancer in adults and may present jointly with a viral hepatitis infection, benign liver tumors (e.g., hepatocellular adenoma) or toxin exposure (e.g., excess alcohol consumption). A subject having hepatocellular carcinoma may further have a chronic liver disease, such as cirrhosis. In an embodiment, a subject having hepatocellular carcinoma may exhibit an increased or decreased level of alpha-fetoprotein or des-gamma carboxyprothrombin. In an embodiment, the subject has hepatocellular carcinoma or is identified as having hepatocellular carcinoma. In an embodiment, the subject having hepatocellular carcinoma is administered a single-stranded oligonucleotide described herein.

Non-Hodgkin's lymphoma (NHL) is a grouping of blood cancers comprising over 60 different forms of lymphoma. NHL has be associated with various causes, including a viral or bacterial infection (Helicobacter pylori infection, HHV-8 infection, Epstein Barr virus infection, hepatitis C viral infection, HIV infection), an autoimmune disease, chemical exposure, or certain medical treatments, e.g., radiation therapy. Current treatment for NHL includes chemotherapy, radiotherapy, and stem cell transplants, each of which may be invasive and/or damaging to healthy tissue. In an embodiment, the subject has NHL or is identified as having NHL. In an embodiment, the subject having NHL is administered a single-stranded oligonucleotide described herein.

Bladder cancer refers to a class of cancers associated with the tissues of the urinary bladder, such as transitional cell carcinoma, urachal adenocarcinoma, and squamous cell carcinoma. Common systems include pain with urination, frequent urination, blood in the urine, and lower back pain. Risk factors for bladder cancer include tobacco use, toxin exposure (e.g., dyes, rubbers, plastics), opium consumption, or microbial infection (e.g., Schistosoma infection). In addition, an increased risk for bladder cancer has been associated with mutations in the FGFR3, TP53, PIK3CA, KDM6A, ARID1A, KMT2D, HRAS, TERT, KRAS, CREBBP, RB1, and TSC1 genes. In an embodiment, the subject has bladder cancer or is identified as having bladder cancer. In an embodiment, the subject having bladder cancer is administered a single-stranded oligonucleotide described herein.

Medulloblastoma is an invasive, rapidly growing cerebral neuronal cancer, and is the most common type of brain cancer in children. Medulloblastoma comprises four molecular subgroups (WNT, SHH, group 3 and group 4), each of which may be further subdivided into additional subtypes. For example, SHH medulloblastomas comprise SHHα, SHHβ, SHHγ, and SHHδ. Individuals with mutations in the genes CTNNB1, PTCH1, MLL2, SMARCA4, DDX3X, CTDNEP1, KDM6A, and TBR1 have been shown to have an increased risk of developing medulloblastoma. In some embodiments, the subject has medulloblastoma or is identified as having medulloblastoma. In an embodiment, the subject having medulloblastoma is administered a single-stranded oligonucleotide described herein. In an embodiment, the subject is a child. In an embodiment, the subject is an adult.

Pancreatic adenocarcinoma is the most common form of pancreatic cancer, accounting for roughly 90% of diagnoses each year. Common signs and symptoms of pancreatic adenocarcinoma include unexplained weight loss, loss of appetite, yellow skin, or back pain; however, pancreatic cancer generally is often not diagnosed until after it has spread to other parts of the body. Pancreatic adenocarcinoma originates in the ducts responsible for transporting endocrine secretions out of the organ, often times in the head of the pancreas. Individuals with mutations in the genes KRAS, CDKN2A, p53, and SMAD4 have been shown to have an increased risk of pancreatic adenocarcinoma. The subject may also exhibit symptoms of diabetes, obesity, and chronic pancreatitis. In an embodiment, the subject has pancreatic adenocarcinoma or is identified as having pancreatic adenocarcinoma. In an embodiment, the subject having pancreatic adenocarcinoma is administered a single-stranded oligonucleotide described herein.

In some embodiments, the proliferative disease is associated with a benign neoplasm. For example, a benign neoplasm may include adenoma, fibroma, hemangioma, clonal hematopoiesis, and lipoma. All types of benign neoplasms disclosed herein or known in the art are contemplated as being within the scope of the disclosure.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The single-stranded oligonucleotides and related compositions described herein may be useful in treating and/or preventing a proliferative disease.

### Pharmaceutical Compositions, Administration, and Formulations

The present invention provides single-stranded oligonucleotides suitable for targeting the spliceosome, as well as related compositions thereof. The compositions described herein may be pharmaceutical compositions, e.g., comprising a single-stranded oligonucleotide or a pharmaceutically acceptable salt, solvate, hydrate, tautomer, or stereoisomer, as described herein, and optionally a pharmaceutically acceptable excipient. In certain embodiments, the single-stranded oligonucleotide is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the single-stranded oligonucleotide (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Pharmaceutical compositions provided herein include particle formulations, such as nanoparticle formulations. Particle formulations may be polymer based and/or lipid based.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided oligonucleotide can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a composition required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a single-stranded oligonucleotide for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the a single-stranded oligonucleotide described herein may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a single-stranded oligonucleotide or composition, as described herein, can be administered in combination with one or more additional pharmaceutical agents. The single-stranded oligonucleotides or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The single-stranded oligonucleotide or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or single-stranded oligonucleotide and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or single-stranded oligonucleotide. In some embodiments, the inventive pharmaceutical composition or single-stranded oligonucleotide provided in the container and the second container are combined to form one-unit dosage form.

Thus, in one aspect, provided are kits including a first container comprising a single-stranded oligonucleotide described herein, or a pharmaceutically acceptable salt, solvate, hydrate, tautomer, or stereoisomer thereof, or a pharmaceutical composition thereof. In certain embodiments, the kit of the disclosure includes a first container comprising a single-stranded oligonucleotide described herein, or a pharmaceutically acceptable salt thereof, or a pharmaceutical composition thereof. In certain embodiments, the kits are useful in preventing and/or treating a disease or disorder described herein in a subject (e.g., a proliferative disease). In certain embodiments, the kits further include instructions for administering the single-stranded oligonucleotide, or a pharmaceutically acceptable salt, solvate, hydrate, tautomer, or stereoisomer thereof, or a pharmaceutical composition thereof, to a subject to prevent and/or treat a proliferative disease.

### ENUMERATED EMBODIMENTS

1. A single-stranded oligonucleotide that targets the U1 small nuclear ribonucleic acid (snRNA) comprising one or more of the following properties:
   i) the single-stranded oligonucleotide is between 5 and 40 nucleotides in length;
   ii) the single-stranded oligonucleotide comprises a plurality of internucleotide phosphorothioate linkages;
   iii) the single-stranded oligonucleotide comprises a locked nucleic acid modification; and
   iv) the single-stranded oligonucleotide comprises a 2′O-methyl group or a 2′O-methoxyethyl group.
2. The single-stranded oligonucleotide of embodiment 1, comprising (i).
3. The single-stranded oligonucleotide of any one of the preceding embodiments, comprising (ii).
4. The single-stranded oligonucleotide of any one of the preceding embodiments, comprising (iii).
5. The single-stranded oligonucleotide of any one of the preceding embodiments, comprising (iv).
6. The single-stranded oligonucleotide of any one of the preceding embodiments, wherein the single-stranded oligonucleotide is between 10 and 30 nucleotides in length (e.g., between 13 and 20 nucleotides or between 13 and 18 nucleotides).
7. The single-stranded oligonucleotide of any one of the preceding embodiments, wherein the single-stranded oligonucleotide is between 12 and 20 nucleotides in length (e.g., between 13 and 20 nucleotides or between 13 and 18 nucleotides).
8. The single-stranded oligonucleotide of any one of the preceding embodiments, wherein the single-stranded oligonucleotide comprises at least 3, 4, 6, 8, 10, 12, 13, 14, 15, 16, 17, 18 internucleotide phosphorothioate linkages.
9. The single-stranded oligonucleotide of any one of the preceding embodiments, wherein the single-stranded oligonucleotide comprises a plurality of locked nucleic acid modifications (e.g., at least 2, 3, 4, 5, 6, 7, 8, 9, 10, 11, 12, 13, 14, 15, 16, 17, or 18 locked nucleic acid modifications).
10. The single-stranded oligonucleotide of any one of the preceding embodiments, wherein the single-stranded oligonucleotide comprises a locked nucleic acid modification on the 3′ terminus or the 5′ terminus.
11. The single-stranded oligonucleotide of any one of the preceding embodiments, wherein the single-stranded oligonucleotide comprises at least 1 locked nucleic acid modification (e.g., at least 2, 3, or 4 locked nucleic acid modifications) on the 3′ terminus.
12. The single-stranded oligonucleotide of any one of the preceding embodiments, wherein the single-stranded oligonucleotide comprises at least 1 locked nucleic acid modification (e.g., at least 2, 3, or 4 locked nucleic acid modifications) on the 5′ terminus.
13. The single-stranded oligonucleotide of any one of the preceding embodiments, wherein the single-stranded oligonucleotide comprises a plurality of 2′O-methyl groups (e.g., at least 2, 3, 4, 5, 6, 7, 8, 9, 10, 11, 12, 13, 14, 15, 16, 17, or 18 2′O-methyl groups).
14. The single-stranded oligonucleotide of any one of the preceding embodiments, wherein the single-stranded oligonucleotide comprises a plurality of 2′O-methoxyethyl groups (e.g., at least 2, 3, 4, 5, 6, 7, 8, 9, 10, 11, 12, 13, 14, 15, 16, 17, or 18 2′O-methoxyethyl groups).
15. The single-stranded oligonucleotide of any one of the preceding embodiments, wherein the single-stranded oligonucleotide comprises the sequence GGTA, GTAA, or TAAG.
16. The single-stranded oligonucleotide of any one of the preceding embodiments, wherein the single-stranded oligonucleotide comprises the sequence GGTAA or GTAAG.
17. The single-stranded oligonucleotide of any one of the preceding embodiments, wherein the single-stranded oligonucleotide comprises the sequence GGTAAG.
18. The single-stranded oligonucleotide of any one of the preceding embodiments, wherein the single-stranded oligonucleotide comprises the sequence CCCT, CCTG, CTGC, TGCC, GCCA, CCAG, CAGG, or AGGT.
19. The single-stranded oligonucleotide of any one of the preceding embodiments, wherein the U1 snRNA is a mutant U1 snRNA.
20. The single-stranded oligonucleotide of any one of the preceding embodiments, wherein the U1 snRNA comprises at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more sequence mutations, e.g., as compared to a reference or consensus U1 snRNA sequence.
21. The single-stranded oligonucleotide of any one of the preceding embodiments, wherein the U1 snRNA comprises a mutation at any one of positions 1-4, 7-28, 31-35, 38-50, 56-79, 86-101, 105-109, 112-119, 122-149, and 155-164, e.g., as compared to a reference or consensus U1 snRNA sequence.
22. The single-stranded oligonucleotide of any one of the preceding embodiments, wherein the U1 snRNA comprises a plurality of mutations at any one of positions 1-4, 7-28, 31-35, 38-50, 56-79, 86-101, 105-109, 112-119, 122-149, and 155-164, e.g., as compared to a reference or consensus U1 snRNA sequence.
23. The single-stranded oligonucleotide of any one of the preceding embodiments, wherein the U1 snRNA comprises a mutation at any one of positions 1-4, e.g., as compared to a reference or consensus U1 snRNA sequence.
24. The single-stranded oligonucleotide of any one of the preceding embodiments, wherein the U1 snRNA bears a mutation at position 3, e.g., as compared to a reference or consensus U1 snRNA sequence.
25. A single-stranded oligonucleotide of Formula (I):

X¹-N¹-N²-N³-N⁴-N⁵-N⁶-N⁷-N⁸-N⁹-N¹⁰N¹¹-N¹²-(N¹³)ⱼ-(N¹⁴)ₖ-(N¹⁵)ₗ-(N¹⁶)ₘ-(N¹⁷)ₙ-(N¹⁸)ₒ-(N¹⁹)ₚ-(N²⁰)_{q}-X² (I),
or a pharmaceutically acceptable salt thereof, wherein:
   each of X¹ and X² is independently hydrogen, C₁-C₁₂ alkyl, C₁-C₁₂ heteroalkyl, halo, —OR^{A}, —O—(C₁-C₆ alkyl), —O—(C₁-C₆ heteroalkyl), —N(R^{B})(R^{C}), —C(O)N(R^{B})(R^{C}), —N(R^{B})C(O)R^{D}, a phosphate group (e.g., a monophosphate group, a diphosphate group, a triphosphate group) or a phosphorothioate group;
   each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ is independently absent or nucleotide (e.g., a modified nucleotide or an unmodified nucleotide);
   each of R^{A}, R^{B}, R^{C}, and R^{D} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
   each of j, k, l, m, n, o, p, and q is independently an integer between 0 and 5; and
   x is 0, 1, or 2;
   wherein one or more of N¹, N², N³, N⁴, N⁵, N⁶, N⁷, N⁸, N⁹, N¹⁰, N¹¹, and N¹² are linked together via an internucleotide phosphorothioate linkage; and
   one or more of N¹, N², N³, N⁴, N⁵, N⁶, N⁷, N⁸, N⁹, N¹⁰, N¹¹, and N¹² comprise a locked nucleic acid modification, a 2′O-methyl group, or a 2′O-methoxyethyl group.
26. The single-stranded oligonucleotide of embodiment 25, wherein each of N¹, N², N³, N⁴, N⁵, N⁶, N⁷, N⁸, N⁹, N¹⁰, N¹¹, N¹², N¹³, N¹⁴, N¹, N¹⁶, N¹⁷, N¹⁸, N¹⁹, and N²⁰ is selected from adenosine, cytidine, thymidine, guanosine, and uridine, or a modified form thereof.
27. The single-stranded oligonucleotide of any one of embodiments 25-26, wherein the bond between at least 2, 3, 4, 5, 6, 7, 8, 9, 10, 11, or all of N¹, N², N³, N⁴, N⁵, N⁶, N⁷, N⁸, N⁹, N¹⁰, N¹¹, and N¹² is a phosphorothioate bond.
28. The single-stranded oligonucleotide of any one of embodiments 25-27, wherein one of N¹, N², N³ comprises a locked nucleic acid modification.
29. The single-stranded oligonucleotide of any one of embodiments 25-28, wherein two of N¹, N², N³ comprises a locked nucleic acid modification.
30. The single-stranded oligonucleotide of any one of embodiments 25-29, wherein each of N¹, N², N³ comprises a locked nucleic acid modification.
31. The single-stranded oligonucleotide of any one of embodiments 25-30, wherein one of N¹¹, N¹², N¹³, N¹⁴, N¹, N¹⁶, N¹⁷, and N¹⁸ comprises a locked nucleic acid modification.
32. The single-stranded oligonucleotide of any one of embodiments 25-31, wherein two of N¹¹, N¹², N¹³, N¹⁴, N⁵, N¹⁶, N¹⁷, and N¹⁸ comprises a locked nucleic acid modification.
33. The single-stranded oligonucleotide of any one of embodiments 25-32, wherein each of N¹¹, N¹², N¹³, N¹⁴, N⁵, N¹⁶, N¹⁷, and N¹⁸ comprises a locked nucleic acid modification.
34. The single-stranded oligonucleotide of any one of embodiments 25-33, wherein one of N², N³, N⁴, N⁵, N⁶, N⁷, N⁸, N⁹, N¹⁰, N¹¹, N¹², N¹³, N¹⁴, N⁵, and N¹⁶ independently comprises a 2′O-methyl group.
35. The single-stranded oligonucleotide of any one of embodiments 25-34, wherein 2, 3, 4, 5, 6, 7, 8, 9, 10, 11, 12, 13, or 14 of N², N³, N⁴, N⁵, N⁶, N⁷, N⁸, N⁹, N¹⁰, N¹¹, N¹², N¹³, N¹⁴, N⁵, and N¹⁶ independently comprises a 2′O-methyl group.
36. The single-stranded oligonucleotide of any one of embodiments 25-35, wherein each of N², N³, N⁴, N⁵, N⁶, N⁷, N⁸, N⁹, N¹⁰, N¹¹, N¹², N¹³ independently comprises a 2′O-methyl group.
37. The single-stranded oligonucleotide of any one of embodiments 25-36, wherein one of N², N³, N⁴, N⁵, N⁶, N⁷, N⁸, N⁹, N¹⁰, N¹¹, N¹², N¹³, N¹⁴, N⁵, and N¹⁶ independently comprises a 2′O-methoxyethyl group.
38. The single-stranded oligonucleotide of any one of embodiments 25-37, wherein 2, 3, 4, 5, 6, 7, 8, 9, 10, 11, 12, 13, or 14 of N², N³, N⁴, N⁵, N⁶, N⁷, N⁸, N⁹, N¹⁰, N¹¹, N¹², N¹³, N¹⁴, N⁵, and N¹⁶ independently comprises a 2′O-methoxyethyl group.
39. The single-stranded oligonucleotide of any one of embodiments 25-37, wherein each of N², N³, N⁴, N⁵, N⁶, N⁷, N⁸, N⁹, N¹⁰, N¹¹, N¹², N¹³ independently comprises a 2′O-methoxyethyl group.
40. The single-stranded oligonucleotide of any one of embodiments 25-39, wherein each of p and q is 0.
41. The single-stranded oligonucleotide of any one of embodiments 25-40, wherein each of j, k, l, m, n, and o is 0 or 1.
42. The single-stranded oligonucleotide of any one of embodiments 25-41, wherein each of X¹ and X² is independently hydrogen, —OR^{A}, or —O—(C₁-C₆ alkyl) (e.g., hydrogen).
43. The single-stranded oligonucleotide of any one of embodiments 25-42, wherein N¹ comprises T or C.
44. The single-stranded oligonucleotide of any one of embodiments 25-43, wherein N² comprises T, C, or G.
45. The single-stranded oligonucleotide of any one of the preceding embodiments, wherein the single-stranded oligonucleotide is selected from an oligonucleotide listed in Table 1.
46. The single-stranded oligonucleotide of any one of the preceding embodiments, wherein the single-stranded oligonucleotide is an antisense oligonucleotide.
47. The single-stranded oligonucleotide of any one of the preceding embodiments, wherein binding of the single-stranded oligonucleotide to the U1 snRNA modulates the binding of the U1 snRNA to a target RNA (e.g., a pre-mRNA, mRNA).
48. The single-stranded oligonucleotide of embodiment 46, wherein the modulating of binding of the U1 snRNA to a target RNA comprises a reduction in binding (e.g., by about 5%, 10%, 20%, 25%, 30%, 40%, 50%, 60%, 70%, 75%, 80%, 90%, 95%, 99%, or more).
49. The single-stranded oligonucleotide of any one of the preceding embodiments, wherein binding of the single-stranded oligonucleotide to the U1 snRNA inhibits the binding of the U1 snRNA to a target RNA (e.g., a pre-mRNA, mRNA).
50. The single-stranded oligonucleotide of any one of the preceding embodiments, wherein binding of the single-stranded oligonucleotide to the U1 snRNA mediates the degradation (e.g., RNAse degradation) of a nucleotide (e.g., an RNA-DNA complex).
51. The single-stranded oligonucleotide of any one of the preceding embodiments, wherein the single-stranded oligonucleotide further comprises a conjugated moiety.
52. The single-stranded oligonucleotide of embodiment 51, wherein the conjugated moiety is a protein, e.g., the catalytic domain of ADAR.
53. The single-stranded oligonucleotide of any one of embodiments 51-52, wherein the protein is capable of altering a target RNA sequence (e.g., conversion of an adenosine to inosine).
54. The single-stranded oligonucleotide of any one of the preceding embodiments, wherein the single-stranded oligonucleotide is complementary with a target sequence within the U1 snRNA sequence (e.g., the 5′ splice site recognition sequence).
55. The single-stranded oligonucleotide of embodiment 54, wherein the target sequence in the U1 snRNA comprises the sequence UUACC.
56. The single-stranded oligonucleotide of any one of the preceding embodiments, wherein the target sequence in the U1 snRNA comprises the sequence UUACC.
57. The single-stranded oligonucleotide of any one of the preceding embodiments, wherein the single-stranded oligonucleotide targets (e.g., binds to) a mutation in the U1 snRNA (e.g., a mutation at any one of positions 1-4, 7-28, 31-35, 38-50, 56-79, 86-101, 105-109, 112-119, 122-149, and 155-164, e.g., a mutation at positions 1-4, e.g., a mutation at position 3).
58. The single-stranded oligonucleotide of any one of the preceding embodiments, wherein the single-stranded oligonucleotide comprises a locked nucleic acid modification at the position corresponding to a mutant nucleotide in the U1 snRNA.
59. A pharmaceutical composition comprising the single-stranded oligonucleotide of any one of embodiments 1-58 and a pharmaceutically acceptable excipient.
60. A method of inhibiting the U1 snRNA processing of a target RNA (e.g., an RNA which encodes an ORF, mRNA, or a pre-mRNA) comprising contacting the target RNA with a single-stranded oligonucleotide, wherein the single-stranded oligonucleotide:
   a) has sufficient homology with the target RNA to hybridize under physiological conditions;
   b) mediates cleavage of the target or hinders binding of the target RNA with another polynucleotide (e.g., RNA or DNA), e.g., a U1 snRNA component; and/or
   c) edits a nucleotide within the sequence of the U1 snRNA component.
61. The method of embodiment 60, comprising a).
62. The method of any one of embodiments 60-61, comprising b).
63. The method of any one of embodiments 60-62, comprising c).
64. The method of embodiment 63, wherein the single-stranded oligonucleotide comprises a conjugated moiety (e.g., a conjugated protein, e.g., the catalytic domain of ADAR).
65. The method of any one of embodiments 60-64, comprising one or more of the following properties:
   i) the single-stranded oligonucleotide is between 5 and 40 nucleotides in length;
   ii) the single-stranded oligonucleotide comprises a plurality of internucleotide phosphorothioate linkages;
   iii) the single-stranded oligonucleotide comprises a locked nucleic acid modification; and
   iv) the single-stranded oligonucleotide comprises a 2′O-methyl group or a 2′O-methoxyethyl group.
66. The method of embodiment 65, comprising (i).
67. The method of any one of embodiments 65-66, comprising (ii).
68. The method of any one of embodiments 65-67, comprising (iii).
69. The method of any one of embodiments 65-68, comprising (iv).
70. The method of any one of embodiments 60-69, wherein the single-stranded oligonucleotide of Formula (I):

X¹-N¹-N²-N³-N⁴-N⁵-N⁶-N⁷-N⁸-N⁹-N¹⁰-N¹¹-N¹²-(N¹³)ⱼ-(N¹⁴)ₖ-(N¹⁵)ₗ-(N¹⁶)ₘ-(N¹⁷)ₙ-(N¹⁸)ₒ-(N¹⁹)ₚ-(N²⁰)_{q}-X² (I),
or a pharmaceutically acceptable salt thereof, wherein:
   each of X¹ and X² is independently hydrogen, C₁-C₁₂ alkyl, C₁-C₁₂ heteroalkyl, halo, —OR^{A}, —O—(C₁-C₆ alkyl), —O—(C₁-C₆ heteroalkyl), —N(R^{B})(R^{C}), —C(O)N(R^{B})(R^{C}), —N(R^{B})C(O)R^{D}, a phosphate group (e.g., a monophosphate group, a diphosphate group, a triphosphate group) or a phosphorothioate group;
   each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹ and N²⁰ is independently absent or nucleotide (e.g., a modified nucleotide or an unmodified nucleotide);
   each of R^{A}, R^{B}, R^{C}, and R^{D} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
   each of j, k, l, m, n, o, p, and q is independently an integer between 0 and 5; and
   x is 0, 1, or 2;
   wherein one or more of N¹, N², N³, N⁴, N⁵, N⁶, N⁷, N⁸, N⁹, N¹⁰, N¹¹, and N¹² are linked together via an internucleotide phosphorothioate linkage; and
   one or more of N¹, N², N³, N⁴, N⁵, N⁶, N⁷, N⁸, N⁹, N¹⁰, N¹¹, and N¹² comprise a locked nucleic acid modification, a 2′O-methyl group, or a 2′O-methoxyethyl group.
71. The method of embodiment 70, wherein each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ is selected from adenosine, cytidine, thymidine, guanosine, and uridine, or a modified form thereof.
72. The method of any one of embodiments 70-71, wherein the bond between at least 2, 3, 4, 5, 6, 7, 8, 9, 10, 11, or all of N¹, N², N³, N⁴, N⁵, N⁶, N⁷, N⁸, N⁹, N¹⁰, N¹¹, and N¹² is a phosphorothioate bond.
73. The method of any one of embodiments 70-72, wherein one of N¹, N², N³ comprises a locked nucleic acid modification.
74. The method of any one of embodiments 70-73, wherein two of N¹, N², N³ comprises a locked nucleic acid modification.
75. The method of any one of embodiments 70-74, wherein each of N¹, N², N³ comprises a locked nucleic acid modification.
76. The method of any one of embodiments 70-75, wherein one of N¹¹, N¹², N¹³, N¹⁴, N¹, N¹⁶, N¹⁷, and N¹⁸ comprises a locked nucleic acid modification.
77. The method of any one of embodiments 70-76, wherein two of N¹¹, N¹², N¹³, N¹⁴, N¹, N¹⁶, N¹⁷, and N¹⁸ comprises a locked nucleic acid modification.
78. The method of any one of embodiments 70-77, wherein each of N¹¹, N¹², N¹³, N¹⁴, N¹, N¹⁶, N¹⁷, and N¹⁸ comprises a locked nucleic acid modification.
79. The method of any one of embodiments 70-78, wherein one of N², N³, N⁴, N⁵, N⁶, N⁷, N⁸, N⁹, N¹⁰, N¹¹, N¹², N¹³, N¹⁴, N⁵, and N¹⁶ independently comprises a 2′O-methyl group.
80. The method of any one of embodiments 70-79, wherein 2, 3, 4, 5, 6, 7, 8, 9, 10, 11, 12, 13, or 14 of N², N³, N⁴, N⁵, N⁶, N⁷, N⁸, N⁹, N¹⁰, N¹¹, N¹², N¹³, N¹⁴, N⁵, and N¹⁶ independently comprises a 2′O-methyl group.
81. The method of any one of embodiments 70-80, wherein each of N², N³, N⁴, N⁵, N⁶, N⁷, N⁸, N⁹, N¹⁰, N¹¹, N¹², N¹³ independently comprises a 2′O-methyl group.
82. The method of any one of embodiments 70-81, wherein one of N², N³, N⁴, N⁵, N⁶, N⁷, N⁸, N⁹, N¹⁰, N¹¹, N¹², N¹³, N¹⁴, N¹, and N¹⁶ independently comprises a 2′O-methoxyethyl group.
83. The method of any one of embodiments 70-82, wherein 2, 3, 4, 5, 6, 7, 8, 9, 10, 11, 12, 13, or 14 of N², N³, N⁴, N⁵, N⁶, N⁷, N⁸, N⁹, N¹⁰, N¹¹, N¹², N¹³, N¹⁴, N¹⁵, and N¹⁶ independently comprises a 2′O-methoxyethyl group.
84. The method of any one of embodiments 70-83, wherein each of N², N³, N⁴, N⁵, N⁶, N⁷, N⁸, N⁹, N¹⁰, N¹¹, N¹², N¹³ independently comprises a 2′O-methoxyethyl group.
85. The method of any one of embodiments 70-84, wherein each of p and q is 0.
86. The method of any one of embodiments 70-84, wherein each of j, k, l, m, n, and o is 0 or 1.
87. The method of any one of embodiments 70-86, wherein each of X¹ and X² is independently hydrogen, —OR^{A}, or —O—(C₁-C₆ alkyl) (e.g., hydrogen).
88. The method of any one of embodiments 70-87, wherein N¹ comprises T or C.
89. The method of any one of embodiments 70-88, wherein N² comprises T, C, or G.
90. The method of any one of embodiments 70-89, wherein the single-stranded oligonucleotide comprises a locked nucleic acid modification at the position corresponding to a mutant nucleotide in the U1 snRNA or target RNA.
91. The method of any one of embodiments 70-90, wherein the single-stranded oligonucleotide is selected from an oligonucleotide listed in Table 1.
92. The method of any one of embodiments 70-91, wherein the single-stranded oligonucleotide is an antisense oligonucleotide.
93. The method of any one of embodiments 70-92, wherein binding of the single-stranded oligonucleotide to the U1 snRNA modulates the binding of the U1 snRNA to a target RNA (e.g., a pre-mRNA, mRNA).
94. The method of embodiment 93, wherein the modulating of binding of the U1 snRNA to a target RNA comprises a reduction in binding (e.g., by about 5%, 10%, 20%, 25%, 30%, 40%, 50%, 60%, 70%, 75%, 80%, 90%, 95%, 99%, or more).
95. The method of any one of embodiments 70-94, wherein binding of the single-stranded oligonucleotide to the U1 snRNA inhibits the binding of the U1 snRNA to a target RNA (e.g., a pre-mRNA, mRNA).
96. The method of any one of embodiments 70-95, wherein the single-stranded oligonucleotide is between 12 and 20 nucleotides in length (e.g., between 13 and 20 nucleotides or between 13 and 18 nucleotides).
97. The method of any one of embodiments 70-96, wherein the single-stranded oligonucleotide comprises a locked nucleic acid modification on the 3′ terminus or the 5′ terminus.
98. The method of any one of embodiments 70-97, wherein the single-stranded oligonucleotide comprises a plurality of 2′OMe-substitutions (e.g., at least 2, 3, 4, 5, 6, 7, 8, 9, 10, 11, 12, 13, 14, 15, 16, 17, or 18 2′OMe-substitutions).
99. The method of any one of embodiments 70-98, wherein the single-stranded oligonucleotide comprises the sequence GGTA, GTAA, or TAAG.
100. The method of any one of embodiments 66-99, wherein the single-stranded oligonucleotide comprises the sequence GGTAA or GTAAG.
101. The method of any one of embodiments 70-100, wherein the single-stranded oligonucleotide comprises the sequence GGTAAG.
102. The method of any one of embodiments 70-101, wherein the single-stranded oligonucleotide comprises the sequence CCCT, CCTG, CTGC, TGCC, GCCA, CCAG, CAGG, or AGGT.
103. A method of modulating (e.g., increasing or decreasing) the splicing of a nucleic acid (e.g., DNA, RNA, e.g., a pre-mRNA) comprising contacting the nucleic acid with a single-stranded oligonucleotide according to any one of embodiments 1-58 or a pharmaceutical composition of embodiment 59.
104. A method of forming a complex between the U1 snRNA and a single-stranded oligonucleotide according to any one of embodiments 1-58 or a pharmaceutical composition of embodiment 59, comprising contacting the U1 snRNA (e.g., in a cell or in vitro) with the single-stranded oligonucleotide.
101. A method of treating a disease or disorder in a subject comprising administering to the subject a single-stranded oligonucleotide according to any one of embodiments 1-58 or a pharmaceutical composition of embodiment 59.
102. The method of embodiment 101, wherein the disease or disorder comprises a proliferative disease (e.g., cancer, a benign neoplasm, or angiogenesis).
103. The method of any one of embodiments 101-102, wherein the proliferative disease is cancer.
104. The method of embodiment 103, wherein the cancer is selected from chronic lymphocytic leukemia, hepatocellular carcinoma, non-Hodgkin lymphoma (B cell non-Hodgkin lymphoma), bladder cancer, medulloblastoma, or pancreatic adenocarcinoma.

### EXAMPLES

In order that the embodiments described herein may be more fully understood, the following examples are set forth. The examples described in this application are offered to illustrate the oligonucleotides, compositions, and related methods provided herein and are not to be construed in any way as limiting their scope.

### Example 1: Synthesis of Exemplary Oligonucleotides

Single-stranded oligonucleotides were obtained from a commercial vendor (see Table 1 for exemplary single-stranded oligonucleotides). The design of these sequences was guided by two main approaches. In the RNAse-mediated degradation approach, the single-stranded oligonucleotide sequence was optimized to bind to a target RNA (e.g., the U1 snRNA) and recruit RNAse H, which then may degrade the single-stranded oligonucleotide/target RNA complex. In the steric blocking approach, the single-stranded oligonucleotide sequence was optimized for binding to a mutant U1 snRNA, e.g., to prevent the mutant U1 snRNA from binding to a target mRNA.

### Example 2: Optimization of Cell Transfection Conditions

In optimize cell transfection conditions, two fluorescently labeled control (non-targeting) single-stranded oligonucleotides were prepared and formulated in either RNAi Max solution (Thermo Fisher) or Lipofectamine 3000 (Thermo Fisher) according to the manufacturer's recommendations. These oligonucleotides were then transfected into either the U1 snRNA mutant cell lines MFE319 or JHH7 for 24, 48 or 72 hours at 200, 100, 50, 25, or 12.5 nM. Transfection efficiencies were assessed by fluorescence microscopy, which indicated that a range of oligonucleotide concentrations could be further tested for splicing modulation.

### Example 3: Screening Single-Stranded Oligonucleotides for U1 snRNA Downregulation

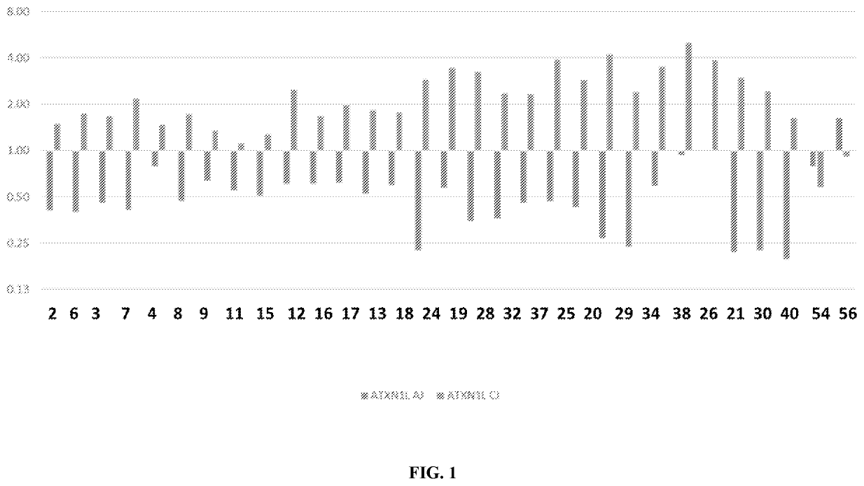

Exemplary single-stranded oligonucleotides and the two non-targeting control sequences were transfected into both MFE319 and JHH7 cell lines for 24 hours using the transfection protocol outlined in Example 2. Treated cells were then washed in PBS, and the Cells to Ct kit (Thermo Fisher) was used to extract the RNA and reverse-transcribe the cDNA from the samples. Three target transcripts (ATXN1L, ACAD10, and CSPP1) were chosen as a proxy for U1 snRNA modulation, as these genes are representative of a larger panel that undergoes alternative splicing in U1 snRNA mutant cancer cell lines as compared to wild type cancer cell lines, and this scheme allowed for more rapid screening of samples. RT-PCT primer and probe pairs were designed for each of three genes, with one set for the alternatively transcribed transcript (AJ=alternative junction) and a second set for the canonical transcript (CJ=canonical junction). Quantitative RT-PCR was then performed to determine which single-stranded oligonucleotides resulted in the greatest efficacy. For example, FIG. 1 displays the modulation of ATXN1L AJ and CJ transcripts. The efficiency of the single-stranded oligonucleotides correlates with an increase in the expression of the canonical junction (CJ) and a decrease in the expression of the alternative junction (AJ). As shown in FIG. 1, several single-stranded oligonucleotides tested at 200 nm concentration (e.g. #20, #21, #24, #29 and #30) showed greater than a two-fold modulation of splicing and thus are appropriate molecules for inhibiting mutant U1 activity.

### Example 4: In Vitro Analysis of U1 snRNA Inhibition

Based on the results from the preliminary screening assay outlined in Example 3, three of the best performing single-stranded oligonucleotides were transfected into for 24 hours. RNA was then extracted from the cell pellets and split into two fractions. The first fraction of RNA was subjected to RT-PCR for the U1 mutant allele (i.e., allele-specific PCR) to confirm that the specific mutant allele was targeted by the single-stranded oligonucleotide, compared to non-targeting controls. The second fraction of RNA was prepped for RNA sequencing using NGS techniques. The data indicated that the single-stranded oligonucleotides were able to revert alternatively spliced pre-mRNA transcripts to patterns more similar to those seen in wild type U1 cancer cell lines.

### Example 5: Effect of Inhibition of U1 Mutants on Cell Proliferation

The effect of U1 mutant allele modulation on cell proliferation was then investigated. The single-stranded oligonucleotides were transfected into either the U1 snRNA mutant cell lines (MFE319 or JHH7 cells) or two control cell lines (wild type for U1 snRNA) for 24, 48, 96, and 144 hours. At each time point, cell viability was monitored using the Cell Titer Glo reagent (Promega). Several single-stranded oligonucleotides tested resulted in a significant modulation of induced a significant decrease in cell viability as compared to the control samples. Further, the single-stranded oligonucleotides had negligible effects on cell viability in the control cell lines. Taken together, these data suggest that inhibition of the U1 mutant splicing using single-stranded oligonucleotides leads to normalization of alternate splicing observed in U1 mutant cell lines and may lead to reduction in cancer cell proliferation. U1 mutant knockout clones were engineered in JHH7 cell line and compared against the parental cell line with and without treatment with oligonucleotides.

1. A single-stranded oligonucleotide that targets the U1 small nuclear ribonucleic acid (snRNA), wherein:
i) the single-stranded oligonucleotide is between 5 and 40 nucleotides in length;
ii) the single-stranded oligonucleotide comprises a plurality of internucleotide phosphorothioate linkages;
iii) the single-stranded oligonucleotide comprises a locked nucleic acid modification; and
iv) the single-stranded oligonucleotide comprises a 2′O-methyl group or a 2′O-methoxyethyl group.

2. The single-stranded oligonucleotide of claim 1, wherein the single-stranded oligonucleotide is between 10 and 30 nucleotides in length (e.g., between 13 and 20 nucleotides or between 13 and 18 nucleotides).

3. The single-stranded oligonucleotide of claim 1, wherein the single-stranded oligonucleotide comprises at least 3, 4, 6, 8, 10, 12, 13, 14, 15, 16, 17, 18 internucleotide phosphorothioate linkages.

4. The single-stranded oligonucleotide of claim 1, wherein the single-stranded oligonucleotide comprises a plurality of locked nucleic acid modifications (e.g., at least 2, 3, 4, 5, 6, 7, 8, 9, 10, 11, 12, 13, 14, 15, 16, 17, or 18 locked nucleic acid modifications).

5. The single-stranded oligonucleotide of claim 1, wherein the single-stranded oligonucleotide comprises a locked nucleic acid modification on the 3′ terminus or the 5′ terminus.

6. The single-stranded oligonucleotide of claim 1, wherein the single-stranded oligonucleotide comprises a plurality of 2′O-methyl groups (e.g., at least 2, 3, 4, 5, 6, 7, 8, 9, 10, 11, 12, 13, 14, 15, 16, 17, or 18 2′O-methyl groups).

7. The single-stranded oligonucleotide of claim 1, wherein the single-stranded oligonucleotide comprises a plurality of 2′O-methoxyethyl groups (e.g., at least 2, 3, 4, 5, 6, 7, 8, 9, 10, 11, 12, 13, 14, 15, 16, 17, or 18 2′O-methoxyethyl groups).

8. The single-stranded oligonucleotide of claim 1, wherein the single-stranded oligonucleotide comprises the sequence GGTA, GTAA, or TAAG.

9. The single-stranded oligonucleotide of claim 1, wherein the single-stranded oligonucleotide comprises the sequence CCCT, CCTG, CTGC, TGCC, GCCA, CCAG, CAGG, or AGGT.

10. The single-stranded oligonucleotide of claim 1, wherein the U1 snRNA is a mutant U1 snRNA.

11. The single-stranded oligonucleotide of claim 1, wherein the U1 snRNA comprises at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more sequence mutations, e.g., as compared to a reference or consensus U1 snRNA sequence.

12. The single-stranded oligonucleotide of claim 1, wherein the U1 snRNA comprises a mutation at any one of positions 1-4, 7-28, 31-35, 38-50, 56-79, 86-101, 105-109, 112-119, 122-149, and 155-164, e.g., as compared to a reference or consensus U1 snRNA sequence.

13. A single-stranded oligonucleotide of Formula (I):
X¹-N¹-N²-N³-N⁴-N⁵-N⁶-N⁷-N⁸-N⁹-N¹⁰-N¹¹-N¹²-(N¹³)ⱼ-(N¹⁴)ₖ-(N¹⁵)ₗ-(N¹⁶)ₘ-(N¹⁷)ₙ-(N¹⁸)ₒ-(N¹⁹)ₚ-(N²⁰)_{q}-X² (I),
or a pharmaceutically acceptable salt thereof, wherein:
each of X¹ and X² is independently hydrogen, C₁-C₁₂ alkyl, C₁-C₁₂ heteroalkyl, halo, —OR^{A}, —O—(C₁-C₆ alkyl), —O—(C₁-C₆ heteroalkyl), —N(R^{B})(R^{C}), —C(O)N(R^{B})(R^{C}), —N(R^{B})C(O)R^{D}, a phosphate group (e.g., a monophosphate group, a diphosphate group, a triphosphate group) or a phosphorothioate group;
each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ is independently absent or nucleotide (e.g., a modified nucleotide or an unmodified nucleotide);
each of R^{A}, R^{B}, R^{C}, and R^{D} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each of j, k, l, m, n, o, p, and q is independently an integer between 0 and 5; and
x is 0, 1, or 2;
wherein one or more of N¹, N², N³, N⁴, N⁵, N⁶, N⁷, N⁸, N⁹, N¹⁰, N¹¹, and N¹² are linked together via an internucleotide phosphorothioate linkage;
one or more of N¹, N², N³, N⁴, N⁵, N⁶, N⁷, N⁸, N⁹, N¹⁰, N¹¹, and N¹² comprise a locked nucleic acid modification, a 2′O-methyl group, or a 2′O-methoxyethyl group; and
the single-stranded oligonucleotide modulates the binding of the U1 snRNA to a target RNA (e.g., a pre-mRNA, mRNA).

14. The single-stranded oligonucleotide of claim 13, wherein each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ is selected from adenosine, cytidine, thymidine, guanosine, and uridine, or a modified form thereof.

15. The single-stranded oligonucleotide of claim 13, wherein the bond between at least 2, 3, 4, 5, 6, 7, 8, 9, 10, 11, or all of N¹, N², N³, N⁴, N⁵, N⁶, N⁷, N⁸, N⁹, N¹⁰, N¹¹, and N¹² is a phosphorothioate bond.

16. The single-stranded oligonucleotide of claim 13, wherein one of N¹, N², N³, N¹¹, N¹², N¹³, N¹⁴, N¹⁵, N¹⁶, N¹⁷, and N¹⁸ comprises a locked nucleic acid modification.

17. The single-stranded oligonucleotide of claim 13, wherein one of N², N³, N⁴, N⁵, N⁶, N⁷, N⁸, N⁹, N¹⁰, N¹¹, N¹², N¹³, N¹⁴, N¹⁵, and N¹⁶ independently comprises a 2′O-methyl group.

18. The single-stranded oligonucleotide of claim 13, wherein one of N², N³, N⁴, N⁵, N⁶, N⁷, N⁸, N⁹, N¹⁰, N¹¹, N¹², N¹³, N¹⁴, N¹⁵, and N¹⁶ independently comprises a 2′O-methoxyethyl group.

19. The single-stranded oligonucleotide of claim 13, wherein each of p and q is 0.

20. The single-stranded oligonucleotide of claim 13, wherein each of j, k, l, m, n, and o is 0 or 1.

21. The single-stranded oligonucleotide of any one of the preceding claims, wherein the single-stranded oligonucleotide is selected from an oligonucleotide listed in Table 1.

22. The single-stranded oligonucleotide of any one of the preceding claims, wherein the single-stranded oligonucleotide is an antisense oligonucleotide.

23. The single-stranded oligonucleotide of claim 13, wherein the modulating of binding of the U1 snRNA to a target RNA comprises a reduction in binding (e.g., by about 5%, 10%, 20%, 25%, 30%, 40%, 50%, 60%, 70%, 75%, 80%, 90%, 95%, 99%, or more).

24. The single-stranded oligonucleotide of claim 13, wherein binding of the single-stranded oligonucleotide to the U1 snRNA inhibits the binding of the U1 snRNA to a target RNA (e.g., a pre-mRNA, mRNA).

25. The single-stranded oligonucleotide of claim 13, wherein binding of the single-stranded oligonucleotide to the U1 snRNA mediates the degradation (e.g., RNAse degradation) of a nucleotide (e.g., an RNA-DNA complex).

26. The single-stranded oligonucleotide of claim 13, wherein the single-stranded oligonucleotide further comprises a conjugated moiety.

27. The single-stranded oligonucleotide of claim 26, wherein the conjugated moiety is a protein, e.g., the catalytic domain of ADAR.

28. The single-stranded oligonucleotide of claim 27, wherein the protein is capable of altering a target RNA sequence (e.g., conversion of an adenosine to inosine).

29. The single-stranded oligonucleotide of claim 13, wherein the single-stranded oligonucleotide is complementary with a target sequence within the U1 snRNA sequence (e.g., the 5′ splice site recognition sequence).

30. The single-stranded oligonucleotide of claim 29, wherein the target sequence in the U1 snRNA comprises the sequence UUACC.

31. The single-stranded oligonucleotide of claim 13, wherein the single-stranded oligonucleotide targets (e.g., binds to) a mutation in the U1 snRNA (e.g., a mutation at any one of positions 1-4, 7-28, 31-35, 38-50, 56-79, 86-101, 105-109, 112-119, 122-149, and 155-164, e.g., a mutation at positions 1-4, e.g., a mutation at position 3).

32. The single-stranded oligonucleotide of claim 13, wherein the single-stranded oligonucleotide comprises a locked nucleic acid modification at the position corresponding to a mutant nucleotide in the U1 snRNA.

33. A pharmaceutical composition comprising the single-stranded oligonucleotide of claim 1 and a pharmaceutically acceptable excipient.

34. A pharmaceutical composition comprising the single-stranded oligonucleotide of claim 13 and a pharmaceutically acceptable excipient.

35. A method of inhibiting the U1 snRNA processing of a target RNA (e.g., an RNA which encodes an ORF, mRNA, or a pre-mRNA) comprising contacting the target RNA with a single-stranded oligonucleotide, wherein the single-stranded oligonucleotide:
a) has sufficient homology with the target RNA to hybridize under physiological conditions;
b) mediates cleavage of the target or hinders binding of the target RNA with another polynucleotide (e.g., RNA or DNA), e.g., a U1 snRNA component; and/or
c) edits a nucleotide within the sequence of the U1 snRNA component.

36. The method of claim 35, comprising a).

37. The method of claim 35, comprising b).

38. The method of claim 35, comprising c).

39. The method of claim 35, wherein the single-stranded oligonucleotide comprises a conjugated moiety (e.g., a conjugated protein, e.g., the catalytic domain of ADAR).

40. The method of claim 35, comprising one or more of the following properties:
i) the single-stranded oligonucleotide is between 5 and 40 nucleotides in length;
ii) the single-stranded oligonucleotide comprises a plurality of internucleotide phosphorothioate linkages;
iii) the single-stranded oligonucleotide comprises a locked nucleic acid modification; and
iv) the single-stranded oligonucleotide comprises a 2′O-methyl group or a 2′O-methoxyethyl group.

41. The method of claim 40, comprising (i).

42. The method of claim 40, comprising (ii).

43. The method of claim 40, comprising (iii).

44. The method of claim 40, comprising (iv).

45. The method of claim 40, wherein the single-stranded oligonucleotide of Formula (I):
X¹-N¹-N²-N³-N⁴-N⁵-N⁶-N⁷-N⁸-N⁹-N¹⁰-N¹¹-N¹²-(N¹³)ⱼ-(N¹⁴)ₖ-(N¹⁵)ₗ-(N¹⁶)ₘ-(N¹⁷)ₙ-(N¹⁸)ₒ-(N¹⁹)ₚ-(N²⁰)_{q}-X² (I),
or a pharmaceutically acceptable salt thereof, wherein:
each of X¹ and X² is independently hydrogen, C₁-C₁₂ alkyl, C₁-C₁₂ heteroalkyl, halo, —OR^{A}, —O—(C₁-C₆ alkyl), —O—(C₁-C₆ heteroalkyl), —N(R^{B})(R^{C}), —C(O)N(R^{B})(R^{C}), —N(R^{B})C(O)R^{D}, a phosphate group (e.g., a monophosphate group, a diphosphate group, a triphosphate group) or a phosphorothioate group;
each of N¹, N², N³, N⁴, N⁵, N⁶, N⁷, N⁸, N⁹, N¹⁰, N¹¹, N¹², N¹³, N¹⁴, N¹⁵, N¹⁶, N¹⁷, N¹⁸, N¹⁹, and N²⁰ is independently absent or nucleotide (e.g., a modified nucleotide or an unmodified nucleotide);
each of R^{A}, R^{B}, R^{C}, and R^{D} is independently hydrogen, C₁-C₆ alkyl, C₂-C₆ alkenyl, C₂-C₆ alkynyl, C₁-C₆ heteroalkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each of j, k, l, m, n, o, p, and q is independently an integer between 0 and 5; and
x is 0, 1, or 2;
wherein one or more of N¹, N², N³, N⁴, N⁵, N⁶, N⁷, N⁸, N⁹, N¹⁰, N¹¹, and N¹² are linked together via an internucleotide phosphorothioate linkage; and
one or more of N¹, N², N³, N⁴, N⁵, N⁶, N⁷, N⁸, N⁹, N¹⁰, N¹¹, and N¹² comprise a locked nucleic acid modification, a 2′O-methyl group, or a 2′O-methoxyethyl group.

46. The method of claim 45, wherein the single-stranded oligonucleotide is selected from an oligonucleotide listed in Table 1.

47. The method of any one of claim 35, wherein the single-stranded oligonucleotide is an antisense oligonucleotide.

48. The method of claim 35, wherein binding of the single-stranded oligonucleotide to the U1 snRNA modulates the binding of the U1 snRNA to a target RNA (e.g., a pre-mRNA, mRNA).

49. The method of claim 48, wherein the modulating of binding of the U1 snRNA to a target RNA comprises a reduction in binding (e.g., by about 5%, 10%, 20%, 25%, 30%, 40%, 50%, 60%, 70%, 75%, 80%, 90%, 95%, 99%, or more).

50. The method of claim 48, wherein binding of the single-stranded oligonucleotide to the U1 snRNA inhibits the binding of the U1 snRNA to a target RNA (e.g., a pre-mRNA, mRNA).

51. The method of claim 35, wherein the single-stranded oligonucleotide is between 12 and 20 nucleotides in length (e.g., between 13 and 20 nucleotides or between 13 and 18 nucleotides).

52. The method of claim 35, wherein the single-stranded oligonucleotide comprises a locked nucleic acid modification on the 3′ terminus or the 5′ terminus.

53. The method of claim 35, wherein the single-stranded oligonucleotide comprises a plurality of 2′OMe-substitutions (e.g., at least 2, 3, 4, 5, 6, 7, 8, 9, 10, 11, 12, 13, 14, 15, 16, 17, or 18 2′OMe-substitutions).

54. The method of claim 35, wherein the single-stranded oligonucleotide comprises the sequence GGTA, GTAA, or TAAG.

55. The method of claim 35, wherein the single-stranded oligonucleotide comprises the sequence CCCT, CCTG, CTGC, TGCC, GCCA, CCAG, CAGG, or AGGT.

56. A method of modulating (e.g., increasing or decreasing) the splicing of a nucleic acid (e.g., DNA, RNA, e.g., a pre-mRNA) comprising contacting the nucleic acid with a single-stranded oligonucleotide according to any one of claim 1 or 13, or a pharmaceutical composition thereof.

57. A method of forming a complex between the U1 snRNA and a single-stranded oligonucleotide according to any one of claim 1 or 13 or a pharmaceutical composition thereof, comprising contacting the U1 snRNA (e.g., in a cell or in vitro) with the single-stranded oligonucleotide.

58. A method of treating a disease or disorder in a subject comprising administering to the subject a single-stranded oligonucleotide according to any one of claim 1 or 13, or a pharmaceutical composition thereof.

59. The method of claim 58, wherein the disease or disorder comprises a proliferative disease (e.g., cancer, a benign neoplasm, or angiogenesis).

60. The method of claim 59, wherein the proliferative disease is cancer.

61. The method of claim 60, wherein the cancer is selected from chronic lymphocytic leukemia, hepatocellular carcinoma, non-Hodgkin lymphoma (B cell non-Hodgkin lymphoma), bladder cancer, medulloblastoma, or pancreatic adenocarcinoma.
